(19) United States
(12) Patent Application Publication
(10) Pub. No.: US 2025/0302622 A1
(43) Pub. Date: Oct. 2, 2025

(54) ANCHOR-STORAGE DEVICES

(71) Applicant: EDWARDS LIFESCIENCES CORPORATION, Irvine, CA (US)

(72) Inventor: Matthew Aaron Sussman, Laguna Hills, CA (US)

(21) Appl. No.: 19/240,535

(22) Filed: Jun. 17, 2025

Related U.S. Application Data
(63) Continuation of application No. PCT/US2023/084131, filed on Dec. 14, 2023.
(60) Provisional application No. 63/433,906, filed on Dec. 20, 2022.
(60) Provisional application No. 63/497,435, filed on Apr. 20, 2023.

(51) Int. Cl. A61F 2/24

(52) U.S. Cl. CPC A61F 2/2445; A61F 2/2466; A61F 2220/0016

(57) ABSTRACT

A system comprises a series of anchors, an anchor-storage device, and an anchor driver that is slidably coupled to the anchor-storage device. The anchor-storage device comprises a spring, and a housing, shaped to define: (i) a loading site, and (ii) a spiral channel that spirals inward to the loading site, the series of anchors arranged along the spiral channel, and the spring configured to urge the series of anchors along the spiral channel toward the loading site. The anchor driver comprises a rod extendable through the loading site in a manner that obstructs access of the series of anchors to the loading site from the spiral channel. Other embodiments are also described.

### CROSS-REFERENCE TO RELATED APPLICATIONS

This application is a continuation of International Patent Application No. PCT/US2023/084131, filed Dec. 14, 2023, which claims the benefit of U.S. Patent Application No. 63/433,906, filed Dec. 20, 2022, and of U.S. Patent Application No. 63/497,435, filed Apr. 20, 2023, the entire disclosures all of which are incorporated by reference for all purposes.

### BACKGROUND

Tissue anchors may be placed intracorporeally so as to anchor an implant to a tissue of a subject. Such tissue anchors may be sufficiently small to be advanced transcatheterally. Due to their small size, they may benefit from a system or apparatus that reduces manual dexterity required to handle the anchors. Furthermore, it may be advantageous to maintain sterility of the anchors. It therefore may be advantageous to provide devices and techniques to facilitate storage and handling of such tissue anchors.

### SUMMARY

In some implementations, methods and systems of housing and utilizing tissue anchors during a transluminal procedure in which the anchors are implanted within a body lumen (e.g., within a heart) of a subject are disclosed. The body lumen and/or the subject may be of a living subject or of a simulation.

In some implementations, a series of tissue anchors can be anchored to tissue (e.g., of a heart, of a heart valve, of a muscle, etc.), e.g., in order to anchor an implant to the tissue. In some implementations, an anchor driver of a delivery assembly (e.g., a drive head at the distal end of the anchor driver) can, for each anchor of the series sequentially, engage the anchor, advance the anchor through the delivery assembly (e.g., through a catheter thereof), and anchor the anchor to tissue.

In some implementations, for each anchor of the series, once the anchor is anchored to the tissue, the drive head is disengaged from the anchor, and the anchor driver is withdrawn (leaving the anchor implanted within the tissue) to allow for the anchor driver to engage with the next anchor of the series.

In some implementations, the systems, apparatuses, devices, etc. herein include an anchor-storage device that facilitates such an iterative anchoring process but, advantageously, without requiring that the drive head be removed out of the delivery assembly.

In accordance with some implementations, a system and/or an apparatus includes a series of anchors and an anchor-storage device.

In some implementations, the anchor-storage device includes a spring and a housing.

In some implementations, the housing is shaped to define: (i) a loading site, and (ii) a spiral channel that spirals inward to the loading site, the series of anchors arranged along the spiral channel, the spring configured to urge the series of anchors along the spiral channel toward the loading site.

In some implementations, the system/apparatus further includes an anchor driver, slidably coupled to the anchor-storage device, and including a rod extendable through the loading site in a manner that obstructs access of the series of anchors to the loading site from the spiral channel.

In some implementations, the system/apparatus includes an implant, the implant including the series of anchors, and a tether that extends between the anchors of the series.

In some implementations, the spring lies along a portion of the channel.

In some implementations, the anchor driver defines a drive head at a distal end of the rod.

In some implementations, for each anchor of the series, while the anchor is disposed in the loading site, the anchor is configured to be engaged by the drive head.

In some implementations, the anchor driver is configured, while the drive head remains engaged with the anchor, to transluminally advance the anchor to a heart of a subject by advancing the rod distally through the loading site.

In some implementations, the anchor driver is configured, while the rod remains extended through the loading site, to anchor the anchor to tissue of the heart.

In some implementations, the system/apparatus further includes an anchor-release mechanism at a proximal end of the system/apparatus, adapted to release the anchor from the drive head once the anchor is anchored to the tissue.

In some implementations, the urging of the series of anchors by the spring automatically moves an anchor of the series into the loading site upon withdrawal of the rod from the loading site.

In some implementations, the spring is disposed within the spiral channel, and is configured to automatically expand within the channel upon withdrawal of the rod from the loading site.

In some implementations, the anchor driver defines a drive head at a distal end of the rod. In some implementations, the housing defines a proximal stop that obstructs the drive head from exiting the anchor-storage device proximally.

In some implementations, the rod is slidable through the proximal stop.

In some implementations, the system/apparatus further includes a driver spring disposed within the housing.

In some implementations, the anchor-storage device is configured such that withdrawing the anchor driver proximally out of the loading site compresses the driver spring against the proximal stop.

In some implementations, the driver spring is a helical compression spring.

In some implementations, the anchor driver further defines a lateral protrusion at a distal portion of the rod. In some implementations, the protrusion is dimensioned such that withdrawing the anchor driver proximally out of the loading site causes the protrusion to compress the driver spring against the proximal stop.

In some implementations, the anchor driver includes a ring that is mounted on the distal portion of the rod, and that defines the protrusion.

In some implementations, the anchor-storage device further includes a cuff threaded onto the rod, and reversibly positionable within the loading site.

In some implementations, the protrusion and the cuff are shaped and positioned such that withdrawing the anchor driver proximally out of the loading site causes the protrusion to force the cuff proximally to compress the driver spring against the proximal stop.

In some implementations, the anchor driver is extendable through the loading site in a manner that positions the cuff within the loading site in a manner that obstructs access of the series of anchors to the loading site from the spiral channel.

In some implementations, the driver spring is configured to push the drive head to the anchor at the loading site.

In some implementations, the drive head is configured to automatically lock onto the anchor upon being pushed to the anchor by the driver spring.

In some implementations, the housing defines an opening in communication with the loading site, configured to allow the anchor driver to advance each anchor distally out of the loading site.

In some implementations, the spiral channel circumscribes an axis, and the opening is configured to allow the anchor driver to advance each anchor out of the loading site distally along the axis.

In some implementations, the spring arcs around axis.

In some implementations, the anchor driver has a drive head at distal end of the rod. In some implementations, for each anchor: (i) the drive head is configured to engage the anchor, and (ii) the anchor driver is configured to transluminally anchor the anchor to tissue of a heart of a subject by, while the rod remains extended through the loading site, advancing the drive head and the anchor distally through the opening, out of the anchor-storage device, and transluminally to the heart.

In some implementations, the system/apparatus further includes a series of cartridges, each cartridge of the series housing a corresponding anchor of the series, and being arranged along the spiral channel.

In some implementations, for each cartridge of the series, the housing is configured to retain the cartridge while the anchor driver advances the corresponding anchor distally out of the anchor-storage device.

In some implementations, the housing defines a receptacle that is in communication with the loading site via the opening, and that is dimensioned to retain each cartridge of the series within the receptacle.

In some implementations, the receptacle is positioned distally to the spiral channel.

In some implementations, the receptacle defines a backplate that defines an aperture that, for each anchor of the series, is dimensioned to allow the anchor, but not the corresponding cartridge, to pass distally out of the housing.

In some implementations, each cartridge of the series is shaped to remain threaded on the rod, within the receptacle, while the anchor driver advances the corresponding anchor distally out of the anchor-storage device.

In some implementations, each cartridge of the series is shaped such that withdrawing the anchor driver proximally out of the receptacle unthreads the cartridge from the rod such that the cartridge is retained within the receptacle.

In some implementations, the system/apparatus further includes a catheter, coupled to the housing and extending distally away from the housing.

In some implementations, the anchor driver is configured to transluminally anchor the anchor to tissue of a heart of a subject by, while the rod remains extended through the loading site, advancing the drive head and the anchor distally through the opening, out of the anchor-storage device, and through the catheter to the heart.

In some implementations, the system/apparatus further includes a catheter control handle, adapted to facilitate transluminal steering of the catheter to the heart.

In some implementations, the catheter control handle is positioned axially between the anchor-storage device and the catheter.

In accordance with some implementations, a method (which can be used with tissue, e.g., of a living subject or of a simulation), the method including advancing a first anchor distally out of a loading site defined by a housing of an anchor-storage device, and transluminally to a tissue of a subject.

In some implementations, advancing the first anchor distally out of a loading site is achieved by sliding, distally through the loading site, an anchor driver engaged with the anchor.

In some implementations, the housing defines a spiral channel that spirals inward to the loading site.

In some implementations, the method further includes subsequently, within the subject, disengaging the anchor driver from the anchor.

In some implementations, the method further includes subsequently withdrawing the anchor driver proximally through and out of the loading site such that the anchor-storage device automatically advances a second anchor from the spiral channel into the loading site.

In some implementations, the anchor driver includes a drive head. In some implementations, sliding the anchor driver engaged with the anchor includes sliding the anchor driver while the drive head is engaged with the anchor.

In some implementations, withdrawing the anchor driver proximally through and out of the loading site includes withdrawing the anchor driver proximally out of the loading site without withdrawing the drive head from the anchor-storage device.

In some implementations, transluminally advancing the first anchor to the tissue includes transluminally advancing the first anchor to the tissue through a catheter.

In some implementations, the method further includes actively steering the catheter towards the tissue via vasculature of the subject.

In some implementations, withdrawing the anchor driver proximally through and out of the loading site includes withdrawing the anchor driver proximally out of the loading site such that a driver spring becomes compressed within the anchor-storage device.

In some implementations, the method further includes, subsequently to the withdrawing, releasing the anchor driver such that the driver spring pushes the anchor driver towards the second anchor in the loading site.

In some implementations, releasing the anchor driver such that the driver spring pushes the anchor driver towards the second anchor in the loading site includes releasing the anchor driver such that the anchor driver locks onto the second anchor.

In some implementations, the first anchor is housed in a cartridge, and transluminally advancing the first anchor to the tissue includes advancing the first anchor out of the cartridge and the anchor-storage device while the cartridge remains within the anchor-storage device.

In some implementations, transluminally advancing the first anchor to the tissue includes transluminally advancing the first anchor to the tissue by sliding the anchor driver through the cartridge.

In some implementations, advancing the first anchor out of the cartridge and the anchor-storage device includes advancing the first anchor out of the cartridge and the anchor-storage device while the cartridge remains within the anchor-storage device, threaded on the anchor driver.

In some implementations, the method further includes, subsequently to disengaging the anchor driver from the anchor, unthreading the anchor driver from the cartridge by withdrawing the anchor driver proximally out of the cartridge.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a system and/or an apparatus, includes a series of anchors, an anchor-storage device, and an anchor driver. In some implementations, the anchor-storage device includes a spring and/or a housing.

In some implementations, the housing is shaped to define: (i) a loading site, and/or (ii) a spiral channel that spirals inward to the loading site, the series of anchors arranged along the spiral channel, the spring configured to urge the series of anchors along the spiral channel toward the loading site.

In some implementations, the anchor driver is slidably coupled or couplable to the anchor-storage device, and/or includes a rod extendable through the loading site in a manner that obstructs access of the series of anchors to the loading site from the spiral channel.

In some implementations, the system/apparatus includes an implant, the implant including the series of anchors, and a tether that extends between the anchors of the series.

In some implementations, the spring lies along a portion of the channel.

In some implementations, the anchor driver defines a drive head at a distal end of the rod, and for each anchor of the series, while the anchor is disposed in the loading site, the anchor is configured to be engaged by the drive head.

In some implementations, the anchor driver is configured, while the drive head remains engaged with the anchor, to transluminally advance the anchor to a heart of a subject by advancing the rod distally through the loading site.

In some implementations, the anchor driver is configured, while the rod remains extended through the loading site, to anchor the anchor to tissue of the heart.

In some implementations, the system/apparatus further includes an anchor-release mechanism at a proximal end of the system/apparatus, adapted to release the anchor from the drive head once the anchor is anchored to the tissue.

In some implementations, the urging of the series of anchors by the spring automatically moves an anchor of the series into the loading site upon withdrawal of the rod from the loading site.

In some implementations, the spring is disposed within the spiral channel, and is configured to automatically expand within the channel upon withdrawal of the rod from the loading site.

In some implementations, the anchor driver defines a drive head at a distal end of the rod.

In some implementations, the housing defines a proximal stop that obstructs the drive head from exiting the anchor-storage device proximally.

In some implementations, the rod is slidable through the proximal stop.

In some implementations, the system/apparatus further includes a driver spring disposed within the housing.

In some implementations, the anchor-storage device is configured such that withdrawing the anchor driver proximally out of the loading site compresses the driver spring against the proximal stop.

In some implementations, the driver spring is a helical compression spring.

In some implementations, the anchor driver further defines a lateral protrusion at a distal portion of the rod, and the lateral protrusion is dimensioned such that withdrawing the anchor driver proximally out of the loading site causes the lateral protrusion to compress the driver spring against the proximal stop.

In some implementations, the anchor driver includes a ring that is mounted on the distal portion of the rod, and that defines the lateral protrusion.

In some implementations, the anchor-storage device further includes a cuff threaded onto the rod, and reversibly positionable within the loading site.

In some implementations, the lateral protrusion and the cuff are shaped and positioned such that withdrawing the anchor driver proximally out of the loading site causes the lateral protrusion to force the cuff proximally to compress the driver spring against the proximal stop.

In some implementations, the anchor driver is extendable through the loading site in a manner that positions the cuff within the loading site in a manner that obstructs access of the series of anchors to the loading site from the spiral channel.

In some implementations, the driver spring is configured to push the drive head to the anchor at the loading site.

In some implementations, the drive head is configured to automatically lock onto the anchor upon being pushed to the anchor by the driver spring.

In some implementations, the housing defines an opening in communication with the loading site, configured to allow the anchor driver to advance each anchor distally out of the loading site.

In some implementations, the spiral channel circumscribes an axis, and the opening is configured to allow the anchor driver to advance each anchor out of the loading site distally along the axis.

In some implementations, the spring arcs around axis.

In some implementations, the anchor driver has a drive head at distal end of the rod.

In some implementations, for each anchor: (i) the drive head is configured to engage the anchor, and/or (ii) the anchor driver is configured to transluminally anchor the anchor to tissue of a heart of a subject by, while the rod remains extended through the loading site, advancing the drive head and the anchor distally through the opening, out of the anchor-storage device, and transluminally to the heart.

In some implementations, the system/apparatus further includes a series of cartridges, each cartridge of the series housing a corresponding anchor of the series, and being arranged along the spiral channel.

In some implementations, for each cartridge of the series, the housing is configured to retain the cartridge while the anchor driver advances the corresponding anchor distally out of the anchor-storage device.

In some implementations, the housing defines a receptacle that is in communication with the loading site via the opening, and that is dimensioned to retain each cartridge of the series within the receptacle.

In some implementations, the receptacle is positioned distally to the spiral channel.

In some implementations, the receptacle defines a backplate that defines an aperture that, for each anchor of the series, is dimensioned to allow the anchor, but not the corresponding cartridge, to pass distally out of the housing.

In some implementations, each cartridge of the series is shaped to remain threaded on the rod, within the receptacle, while the anchor driver advances the corresponding anchor distally out of the anchor-storage device.

In some implementations, each cartridge of the series is shaped such that withdrawing the anchor driver proximally out of the receptacle unthreads the cartridge from the rod such that the cartridge is retained within the receptacle.

In some implementations, the system/apparatus further includes a catheter, coupled to the housing and extending distally away from the housing.

In some implementations, the anchor driver is configured to transluminally anchor the anchor to the tissue of the heart by, while the rod remains extended through the loading site, advancing the drive head and the anchor distally through the opening, out of the anchor-storage device, and through the catheter to the heart.

In some implementations, the system/apparatus further includes a catheter control handle, adapted to facilitate transluminal steering of the catheter to the heart.

In some implementations, the catheter control handle is positioned axially between the anchor-storage device and the catheter.

In accordance with some implementations, a method (e.g., for use with a real or simulated subject) includes advancing a first anchor distally out of a loading site defined by a housing of an anchor-storage device, and transluminally to a tissue of the subject, by sliding, distally through the loading site. In some implementations, an anchor driver is engaged with the anchor. In some implementations, the housing defines a spiral channel that spirals inward to the loading site.

In some implementations, the method includes, subsequently, within the subject, disengaging the anchor driver from the anchor.

In some implementations, the method includes, subsequently, withdrawing the anchor driver proximally through and out of the loading site such that the anchor-storage device automatically advances a second anchor from the spiral channel into the loading site.

In some implementations, the anchor driver includes a drive head.

In some implementations, sliding the anchor driver engaged with the anchor includes sliding the anchor driver while the drive head is engaged with the anchor.

In some implementations, withdrawing the anchor driver proximally through and out of the loading site includes withdrawing the anchor driver proximally out of the loading site without withdrawing the drive head from the anchor-storage device.

In some implementations, transluminally advancing the first anchor to the tissue includes transluminally advancing the first anchor to the tissue through a catheter.

In some implementations, the method further includes actively steering the catheter towards the tissue via vasculature of the subject.

In some implementations, withdrawing the anchor driver proximally through and out of the loading site includes withdrawing the anchor driver proximally out of the loading site such that a driver spring becomes compressed within the anchor-storage device.

In some implementations, the method further includes, subsequently to the withdrawing, releasing the anchor driver such that the driver spring pushes the anchor driver towards the second anchor in the loading site.

In some implementations, releasing the anchor driver such that the driver spring pushes the anchor driver towards the second anchor in the loading site includes releasing the anchor driver such that the anchor driver locks onto the second anchor.

In some implementations, the first anchor is housed in a cartridge, and transluminally advancing the first anchor to the tissue includes advancing the first anchor out of the cartridge and the anchor-storage device while the cartridge remains within the anchor-storage device.

In some implementations, transluminally advancing the first anchor to the tissue includes transluminally advancing the first anchor to the tissue by sliding the anchor driver through the cartridge.

In some implementations, advancing the first anchor out of the cartridge and the anchor-storage device includes advancing the first anchor out of the cartridge and the anchor-storage device while the cartridge remains within the anchor-storage device, threaded on the anchor driver.

In some implementations, the method further includes, subsequently to disengaging the anchor driver from the anchor, unthreading the anchor driver from the cartridge by withdrawing the anchor driver proximally out of the cartridge.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations, a system and/or an apparatus includes a tether, a series of anchors, threaded onto the tether; and/or an anchor-storage device. In some implementations, the anchor-storage device includes a housing, rotatable about an axis, and including a plurality of anchor storage compartments arranged around the axis, each of the anchor storage compartments housing a respective anchor of the series of anchors.

In some implementations, the anchor-storage device includes a backplate defining an aperture through which each anchor of the series is advanceable distally out of the housing.

In some implementations, the anchor-storage device includes a spool having part of the tether wound therearound.

In some implementations, the housing is rotatable relative to the backplate, in a manner that enables alignment of each of the anchor storage compartments, in sequence, to become aligned with the aperture.

In some implementations, the system/apparatus includes an implant, the implant including the tether and the series of anchors.

In some implementations, the system/apparatus further includes an anchor driver, slidably coupled to the anchor-storage device, and including a rod extendable through the aperture to drive an anchor of the series of anchors, disposed in one of said plurality of anchor storage compartments aligned with the aperture, distally out of the anchor-storage device.

In some implementations, the housing is rotatable relative to the backplate when the anchor driver is disengaged from the aperture.

In some implementations, the housing is not rotatable relative to the backplate when the anchor driver extends through the aperture.

In some implementations, the housing is manually rotatable relative to the backplate.

In some implementations, the anchor-storage device further includes an engagement arm, and an exterior surface of the housing includes a ratchet mechanism adapted to be engaged by the engagement arm, to enable measured rotation of the housing relative to the backplate.

In some implementations, the housing is automatically rotatable relative to the backplate.

In some implementations, the anchor driver defines a drive head at a distal end of the rod.

In some implementations, the housing defines a proximal stop that obstructs the drive head from exiting the anchor-storage device proximally.

In some implementations, the rod is slidable through the proximal stop.

In some implementations, the system/apparatus further includes a driver spring disposed within the housing.

In some implementations, the anchor-storage device is configured such that withdrawing the anchor driver proximally out of the aperture in the backplate compresses the driver spring against the proximal stop.

In some implementations, the driver spring is a helical compression spring.

In some implementations, the anchor driver further defines a lateral protrusion at a distal portion of the rod.

In some implementations, the lateral protrusion is dimensioned such that withdrawing the anchor driver proximally out of the aperture in the backplate causes the lateral protrusion to compress the driver spring against the proximal stop.

In some implementations, the anchor driver includes a ring that is mounted on the distal portion of the rod, and that defines the lateral protrusion.

In some implementations, the anchor-storage device further includes a cuff threaded onto the rod, and reversibly positionable within the loading site.

In some implementations, the lateral protrusion and the cuff are shaped and positioned such that withdrawing the anchor driver proximally out of the loading site causes the lateral protrusion to force the cuff proximally to compress the driver spring against the proximal stop.

In some implementations, the driver spring is configured to push the drive head to the anchor aligned with the aperture.

In some implementations, the drive head is configured to automatically lock onto the anchor upon being pushed to the anchor by the driver spring.

In some implementations, the spool is rotatable relative to the backplate.

In some implementations, the spool is rotatable independently of rotation of the housing.

In some implementations, rotation of the spool tensions the tether during delivery of one or more of the series of tissue anchors distally out of the housing.

In some implementations, the spool is functionally associated with a spring, the spring configured rotate the spool thereby to tension the tether.

In some implementations, the anchor driver defines a drive head at a distal end of the rod.

In some implementations, for each anchor of the series, while the anchor is aligned with the aperture in the backplate, the anchor is configured to be engaged by the drive head.

In some implementations, the anchor driver is configured: (i) while the drive head remains engaged with the anchor, to transluminally advance the anchor to a heart of a subject by advancing the rod distally through the aperture in the backplate, and/or (ii) while the rod remains extended through the aperture, to anchor the anchor to tissue of the heart.

In some implementations, the system/apparatus further includes an anchor-release mechanism at a proximal end of the system/apparatus, adapted to release the anchor from the drive head once the anchor is anchored to the tissue.

In some implementations, the system further includes a series of cartridges, each cartridge of the series housing a corresponding anchor of the series, the cartridges being arranged around the axis of the housing.

In some implementations, the system/apparatus further includes a catheter, coupled to the housing and extending distally away from the housing.

In some implementations, the anchor driver is configured to transluminally anchor the anchor to the tissue of the heart by, while the rod remains extended through the aperture in the backplate, advancing the drive head and the anchor distally out of the anchor-storage device, and through the catheter to the heart.

In some implementations, the system/apparatus further includes a catheter control handle, adapted to facilitate transluminal steering of the catheter to the heart.

In accordance with some implementations, a method (e.g., useable or for use with a subject) includes obtaining an anchor storage device including a housing rotatable about an axis, the housing including a plurality of anchor storage compartments arranged around the axis, each of the anchor storage compartments housing a respective anchor of a series of anchors threaded onto a tether.

In some implementations, the anchor storage device further includes a backplate defining an aperture.

In some implementations, the method includes advancing a first anchor of the series of anchors distally out of one of the anchor storage compartments in the housing, via the aperture in the backplate, and transluminally to a tissue of the subject, by sliding, distally through the aperture, an anchor driver engaged with the anchor.

In some implementations, the method includes, subsequently, within the subject, disengaging the anchor driver from the anchor and withdrawing the anchor driver proximally through and out of the aperture.

In some implementations, the method includes rotating the housing about the axis relative to the backplate to align a second anchor of the series with the aperture in the backplate.

In some implementations, the rotating includes manually rotating the housing about the axis.

In some implementations, the anchor driver includes a drive head, and sliding the anchor driver engaged with the anchor includes sliding the anchor driver while the drive head is engaged with the anchor.

In some implementations, withdrawing the anchor driver proximally through and out of the aperture includes withdrawing the anchor driver proximally out of the aperture while withdrawing the drive head from the anchor-storage device.

In some implementations, the rotating includes automatically rotating the housing about the axis.

In some implementations, the anchor driver includes a drive head, and sliding the anchor driver engaged with the anchor includes sliding the anchor driver while the drive head is engaged with the anchor.

In some implementations, withdrawing the anchor driver proximally through and out of the aperture includes withdrawing the anchor driver proximally out of the aperture without withdrawing the drive head from the anchor-storage device.

In some implementations, withdrawing the anchor driver proximally through and out of the loading site includes withdrawing the anchor driver proximally out of the loading site such that a driver spring becomes compressed within the anchor-storage device.

In some implementations, the automatically rotating includes, subsequently to the withdrawing, releasing the anchor driver such that the driver spring rotates the housing to align the second anchor with the aperture.

In some implementations, releasing the anchor driver includes releasing the anchor driver such that the anchor driver locks onto the second anchor aligned with the aperture.

In some implementations, transluminally advancing the first anchor to the tissue includes transluminally advancing the first anchor to the tissue through a catheter.

In some implementations, the method further includes actively steering the catheter towards the tissue via vasculature of the subject.

In some implementations, the first anchor is housed in a cartridge, and transluminally advancing the first anchor to the tissue includes advancing the first anchor out of the cartridge and the anchor-storage device while the cartridge remains within the anchor-storage device.

In some implementations, transluminally advancing the first anchor to the tissue includes transluminally advancing the first anchor to the tissue by sliding the anchor driver through the cartridge.

In some implementations, advancing the first anchor out of the cartridge and the anchor-storage device includes advancing the first anchor out of the cartridge and the anchor-storage device while the cartridge remains within the anchor-storage device, threaded on the anchor driver.

In some implementations, the method further includes, subsequently to disengaging the anchor driver from the anchor, unthreading the anchor driver from the cartridge by withdrawing the anchor driver proximally out of the cartridge.

In some implementations, the method further includes repeating the steps of advancing, disengaging, and rotating, for each anchor in the series of anchors.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the above methods can comprise (or additional methods comprise or consist of) sterilization of one or more systems, devices, apparatuses, components, etc. herein (e.g., with heat, radiation, ethylene oxide, hydrogen peroxide, etc.).

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

### BRIEF DESCRIPTION OF THE DRAWINGS

FIGS. 1 and 2 are schematic illustrations of an example system comprising a delivery assembly that comprises an anchor-storage device adapted to store a series of anchors therewithin, including aesthetic features thereof, in accordance with some implementations;

FIGS. 3A-C, 4A-C, 5A-C, 6A-B, 7A-C, and 8 represent at least some steps in a series of steps that can be performed in order to anchor a series of anchors to tissue of a subject, using the system of FIGS. 1-2, and show some aesthetic features of the system, in accordance with some implementations;

FIGS. 9A-B are schematic illustrations of an implant and of the delivery assembly of FIGS. 1-2 in use with the implant, and also show some aesthetic features, in accordance with some implementations;

FIG. 10 is a schematic perspective illustration of an anchor-delivery system according to some implementations;

FIG. 11 is an exploded view illustration of an anchor-delivery assembly forming part of the system of FIG. 10 according to some implementations;

FIGS. 12A, 12B, and 12C are sectional illustrations of the anchor-delivery assembly according to some implementations; and

FIGS. 13A, 13B, 13C, 13D, and 13E are schematic illustrations of at least some steps of a process of using the anchor-delivery assembly of FIGS. 11-12C for the delivery and implantation of a tissue anchor of a series of tissue anchors, according to some implementations.

### DETAILED DESCRIPTION

The present disclosure relates, inter alia, to methods and systems of housing and utilizing tissue anchors during a transluminal procedure in which the anchors are implanted within a body lumen (e.g., within a heart) of a subject.

In some implementations, an implant can comprise and/or can be anchored using a series of tissue anchors. For example, during an annuloplasty procedure, a series of tissue anchors can be implanted around an annulus of the heart, in order to facilitate subsequent contraction of the annulus.

In some implementations, during such a procedure, an anchor driver of a delivery assembly (e.g., a drive head at the distal end of the anchor driver) can, for each anchor of the series sequentially, engage the anchor, advance the anchor through the delivery assembly (e.g., through a catheter thereof), and anchor the anchor to tissue.

In some implementations, for each anchor of the series, once the anchor is anchored to the tissue, the drive head is disengaged from the anchor, and the anchor driver is withdrawn (leaving the anchor implanted within the tissue) to allow for the anchor driver to engage with the next anchor of the series.

The current disclosure facilitates an iterative anchoring process but, advantageously, one that does not necessitate that the anchor driver be entirely removed from the delivery assembly after the anchoring of each anchor. For example, in some implementations, the next anchor of the series can be positioned for engagement by the anchor driver, and/or a drive head of the anchor driver can engage the next anchor of the series without requiring that the drive head be removed out of the delivery assembly.

Examples of such annuloplasty implants, systems (e.g., delivery tools), and techniques for which the apparatus and aspects of the present disclosure can be utilized, and/or with which the apparatus and aspects of the present disclosure can be combined, mutatis mutandis, include those described in the following publications, each of which is incorporated herein by reference:
U.S. Patent Application Publication No. 2012/0022557 to Cabiri et al.
U.S. Patent Application Publication No. 2014/0309661 to Sheps et al.
U.S. Patent Application Publication No. 2015/0272734 to Sheps et al.
U.S. Patent Application Publication No. 2018/0049875 to Iflah et al.
U.S. Patent Application Publication No. 2021/0145584 to Kasher et al.

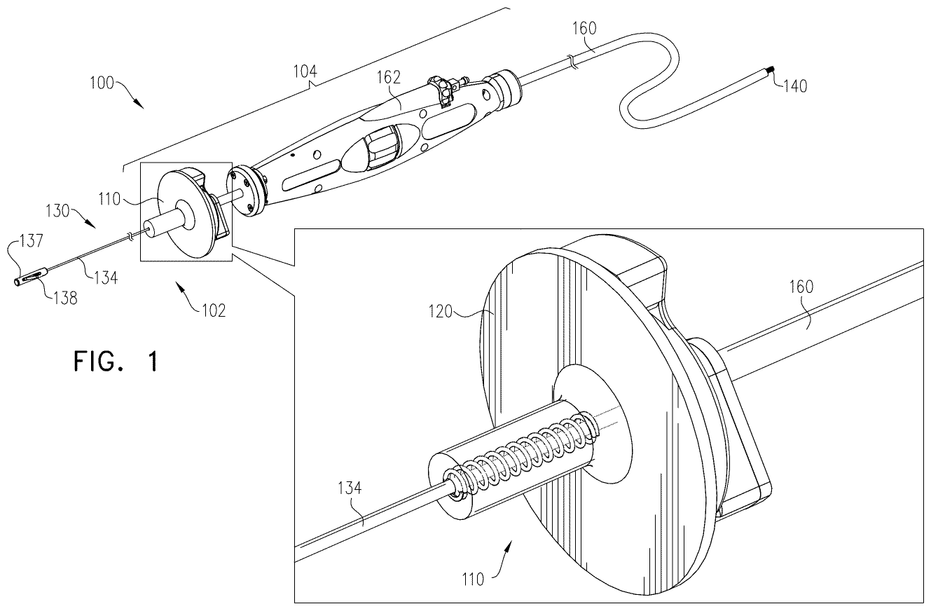

Reference is now made to FIGS. 1, 2, 3A-C, 4A-C, 5A-C, 6A-B, 7A-C, and 8, which are schematic illustrations of an example system 100 comprising a delivery assembly 102 that comprises an anchor-storage device 110 adapted to store a series of anchors 140 therewithin, in accordance with some implementations.

In some implementations, delivery assembly 102 comprises an anchor driver 130, having a rod 134 that is adapted to extend through anchor-storage device 110, and a drive head 136 at a distal end of the rod, adapted to anchor the series of anchors 140 to tissue of a subject while the rod extends through the anchor-storage device.

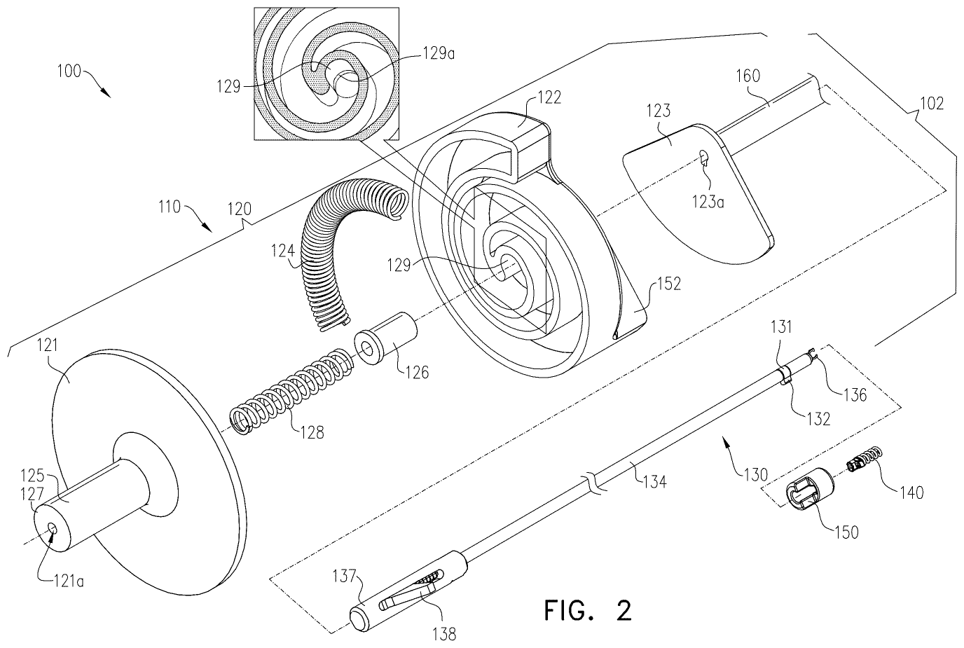

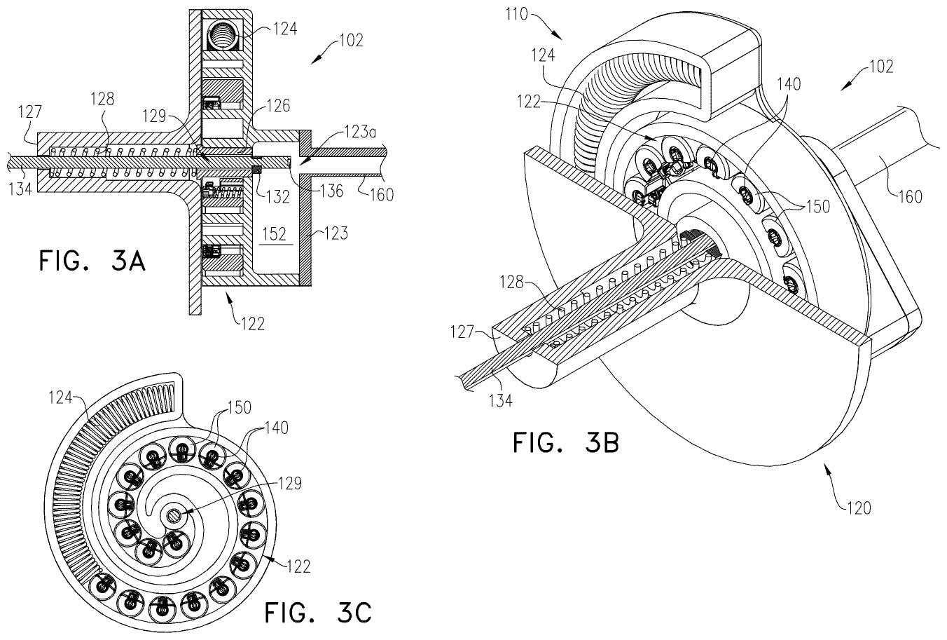

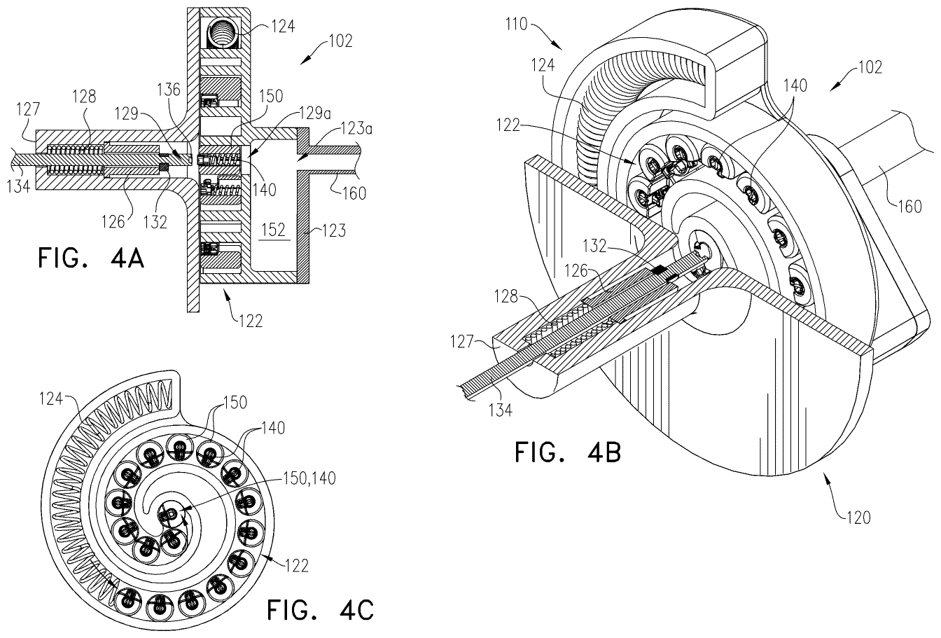

FIG. 1 shows an overall view of system 100. FIG. 2 shows an exploded view of system 100. FIGS. 3A-7C show at least some steps of a series of steps that can be used with system 100. FIG. 8 shows a back view of anchor-storage device 110, after usage. For the sake of clarity, other than in FIG. 6B, catheter handle 162 is omitted from FIGS. 2-8.

In some implementations, delivery assembly 102 comprises a catheter 160, via which anchor driver 130 can transluminally (e.g., transfemorally) advance anchors 140, e.g., to the heart.

In some implementations, a catheter handle 162 is positioned at a proximal part of the catheter (e.g., at a handle portion 104 of the delivery assembly), the catheter handle being adapted to facilitate transluminal advancement of the catheter towards the heart, e.g., by steering a distal steering portion of the catheter via operational coupling (e.g., via pullwires) between the catheter handle and the distal steering portion. Nonlimiting examples of how this steering can be implemented include those described in the following publications, each of which is incorporated by reference:
U.S. Patent Application Publication No. 2014/0309661 to Sheps et al.
U.S. Patent Application Publication No. 2015/0272734 to Sheps et al.

In some implementations, anchor driver 130 comprises an anchor-release mechanism that includes an anchor-release trigger 138 at a proximal part of the anchor driver, e.g., at a driver handle 137 of the anchor driver.

In some implementations, the anchor-release mechanism is adapted to release anchor 140 from drive head 136 once the anchor has been anchored to the tissue. In some implementations, anchor driver 130 and/or its anchor-release mechanism can be as described, mutatis mutandis, in one or more of the following publications, each of which is incorporated herein by reference:
U.S. Patent Application Publication No. 2012/0022557 to Cabiri et al.
U.S. Patent Application Publication No. 2014/0309661 to Sheps et al.
U.S. Patent Application Publication No. 2015/0272734 to Sheps et al.
U.S. Patent Application Publication No. 2018/0049875 to Iflah et al.
U.S. Patent Application Publication No. 2022/0071620 to Brauon et al.
International Patent Application Publication No. WO 2022/064401 to Halabi et al.
International Patent Application No. PCT/IB2022/051099 to Shafigh et al.

In some implementations, and as shown in FIG. 1, anchor-storage device 110 can be positioned at handle portion 104, e.g., proximally from catheter handle 162, such as affixed to a proximal end of the catheter handle.

In some implementations, at least a proximal portion of anchor driver 130 protrudes proximally out of anchor-storage device 110. Thus, at certain stages during use, the anchor driver (e.g., its rod) extends distally through housing 120 and catheter handle 162, and into catheter 160 (e.g., as explained in more detail hereinbelow).

Anchor-storage device 110 will now be described in more detail. In some implementations, anchor-storage device 110 comprises a housing 120, shaped to define a spiral channel 122 that spirals inward towards (e.g., leading to) a loading site 129. For example, the spiral shape of channel 122 can be centered about the loading site, e.g., defining a channel plane. In some implementations, spiral channel 122 serves as an anchor storage compartment for storing anchors during use of anchor-storage device 110.

In some implementations, the series of anchors 140 is arranged along the spiral channel around loading site 129, e.g., as shown. In some implementations, anchor-storage device 110 comprises a spring 124, configured to urge the series of anchors along the spiral channel toward the loading site 129.

In some implementations, during anchoring of each anchor of the series of anchors to the heart, rod 134 extends through loading site 129 in a manner that obstructs access of the series of anchors to the loading site from the spiral channel.

In some implementations, withdrawing rod 134 from loading site 129 can automatically trigger anchor-storage device 110 to move an anchor (e.g., the next anchor) of the series into the loading site. For example, spring 124 can be disposed within the spiral channel in a manner in which withdrawing the rod from the loading site allows the spring to expand within the channel, thereby pushing the series of anchors along the channel such and thereby pushing the next anchor of the series into the loading site.

In some implementations, and as explained in more detail hereinbelow, from this withdrawn state of anchor driver 130, the anchor driver (e.g., drive head 136 thereof) can engage with the next anchor 140 of the series by the anchor driver being returned distally towards loading site 129, such that the drive head contacts and engages with the anchor, after-which the anchor can be advanced, via the anchor driver, through loading site 129 and catheter 160 and towards the heart.

In some implementations, housing 120 defines a front plate 121 that covers spiral channel 122 (and therefore anchors 140 within the channel) and retains the anchors within the channel.

In some implementations, housing 120 defines a proximal stop 127 that obstructs drive head 136 from exiting anchor-storage device 110 proximally.

In some implementations, anchor-storage device 110 comprises a driver spring 128 that is positioned and dimensioned to become strained (e.g., compressed) by anchor driver 130 (e.g., drive head 136 thereof) being retracted proximally from loading site 129, and to thereby bias the driver to return distally toward the loading site.

In some implementations, driver spring 128 can be positioned within a cavity 125 defined by housing 120. In the example shown, driver spring 128 is a helical compression spring, transitionable towards a compressed state by withdrawing anchor driver 130 out from loading site 129 such that the anchor driver compresses the driver spring against proximal stop 127.

In some implementations, in order to maintain the driver spring in the compressed state, a proximal pulling force must be maintained on the anchor driver, to prevent the driver spring from relaxing and pushing the anchor driver towards the loading site. In some implementations, such a pulling force is not necessary.

In some implementations, in order for the anchor driver 130 to engage with the next anchor 140 of the series (e.g., in order to lock drive head 136 with the anchor that is disposed in loading site 129), the operator releases the anchor driver (e.g., ceases to apply the proximal pulling force on the anchor driver), thereby allowing driver spring 128 to relax (e.g., to expand) in a manner that pushes anchor driver 130 towards the anchor 140 that is currently positioned within loading site 129. (Note, this anchor would have arrived at the loading site upon the anchor driver having been withdrawn from the loading site, as described hereinabove.)

In some implementations, the driver spring can be configured to push the anchor driver towards the anchor that is disposed within the loading site in a manner in which the drive head becomes locked onto the anchor. For example, the amount of force applied by the driver spring can be greater than that needed to engage drive head 136 with anchor 140. For example, drive head 136 can snap-fit into engagement with the anchor.

In some implementations, in order to transition the driver spring towards the compressed state, the anchor driver defines a lateral protrusion 132 at a distal portion of rod 134 (e.g., just proximally to drive head 136), e.g., as shown in FIG. 2.

In some implementations, anchor driver 130 comprises a ring 131 that is mounted on the distal portion of the rod, that defines protrusion 132. Ring 131 can be rotatable around rod 134.

In some implementations, the lateral protrusion is dimensioned to transition the driver spring towards the compressed state in response to withdrawing anchor driver 130 from loading site 129. For example, and as shown, a cuff 126 can be threaded onto rod 134, and can be reversibly positionable within loading site 129.

In some implementations, as anchor driver 130 is retracted proximally out of loading site 129, protrusion 132 abuts cuff 126, forcing the cuff proximally out of the loading site, and against driver spring 128, thereby compressing the driver spring against proximal stop 127.

In some implementations, the cuff is dimensioned to obstruct access of the series of anchors to the loading site from the spiral channel while the rod extends through the loading site.

In some implementations, system 100 further comprises a series of cartridges (e.g., capsules) 150, each anchor of the series being housed in a cartridge of the series within spiral channel 122.

In some implementations, channel 122 and cartridges 150 are complimentarily keyed in order to orient the cartridges in a specific way, e.g., such that anchors 140 arrive at loading site 129 in a rotational orientation optimized for locking to drive head 136. For example, channel 122 can define a groove and each cartridge 150 can define a tongue that rides within the groove, and/or the channel can define a rail and each cartridge can define a notch that rides on the rail. In some implementations, each anchor 140 can fit snugly within its corresponding cartridge 150, e.g., such that the anchor is prevented from rotating with respect to its cartridge.

In some implementations, for each cartridge of the series, housing 120 is configured to retain the cartridge while the anchor driver advances the corresponding anchor distally out of the anchor-storage device (e.g., the housing prevents the cartridge from being advanced along with the anchor towards the heart).

Furthermore, housing 120 can be configured to retain each cartridge 150 even after its anchor has been anchored to tissue and driver 130 has been retracted, e.g., once that cartridge is no longer required. For example, in some implementations, housing 120 defines a receptacle 152 (e.g., a disposal area) that is dimensioned to receive the cartridges. Receptacle 152 can be positioned distally to spiral channel 122. Receptacle 152 can be in communication with loading site 129, e.g., via an opening 129a.

The retention of each cartridge 150 within housing 120 while anchor driver 130 drives its anchor 140 distally out of the anchor-storage device can be facilitated by a backplate 123 of receptacle 152 defining an aperture 123a that is shaped and/or dimensioned to allow anchors 140, but not cartridges 150, to pass therethrough. Thus, while anchor driver 130 advances the corresponding anchor 140 distally out of anchor-storage device 110, rod 134 slides through cartridge 150 (e.g., the cartridge becomes threaded around the rod). In some implementations, withdrawing anchor driver 130 proximally back through aperture 123a and into loading site 129 unthreads cartridge 150 from the rod such that the cartridge becomes deposited (e.g., disposed of) within the receptacle.

FIGS. 3A-C, 4A-C, 5A-C, 6A-B, 7A-C, and 8 represent at least some steps in a series of example steps that can be performed in order to anchor a series of anchors 140 to tissue of a subject, using system 100, in accordance with some implementations. It is to be noted that these figures are also intended to illustrate the functionality and capability of system 100, and not merely to define a strict sequence of steps of a procedure. For each of FIGS. 3A-C, 4A-C, 5A-C, and 7A-C, multiple views are provided, with the respective Fig. A (e.g., FIG. 3A) showing a longitudinal cross-section, Fig. C (e.g., FIG. 3C) showing a transverse cross-section (e.g., transverse to the cross-section of Fig. A), and Fig. B (e.g., FIG. 3B) showing a perspective view with part of housing 120 cut away, and part of system 100 (e.g., anchor driver 130 and driver spring 128) in cross-section.

In some implementations, anchor driver 130 (e.g., drive head 136 thereof) is successively engaged with each anchor of the series that is successively loaded into loading site 129, and subsequently used to advance that anchor to the tissue while the anchor driver is extended through the anchor-storage device.

FIGS. 3A-C show anchor-storage device 110 before anchor driver 130 has engaged with any of the anchors of the series. For example, system 100 can be provided in this state. In some implementations, anchor driver 130 extends through anchor-storage device 110 (e.g., rod 134 extends through loading site 129 and distally through housing 120), such that the series of anchors is obstructed from accessing the loading site from the spiral channel. As noted hereinabove, this obstruction can be facilitated by cuff 126, which can be disposed within loading site 129 in this state.

In some implementations, in order to load an anchor (e.g., the first anchor) of the series into loading site 129, anchor driver 130 is then pulled proximally (e.g., by the operator pulling handle 137 proximally), such that anchor driver 130 and/or cuff 126 no longer obstructs access of the series of anchors to the loading site from the spiral channel (FIGS. 4A-C). The anchor 140 thus moves into the loading site, urged forward by spring 124, e.g., as described hereinabove. In some implementations, and as described hereinabove, pulling the anchor driver proximally out of loading site 129 compresses driver spring 128 against proximal stop 127 (e.g., by protrusion 132 contacting cuff 126 and pulling the cuff against the driver spring), e.g., as shown in FIGS. 4A-B.

In some implementations, in order to engage anchor driver 130 with the anchor 140 that is now disposed within loading site 129 (e.g., to lock drive head 136 onto the anchor), anchor driver 130 is then advanced towards that anchor (as shown in the transition from FIGS. 4A-C to FIGS. 5A-C). This can be accomplished by the operator simply releasing anchor driver 130 (e.g., releasing handle 137), such that driver spring 128 pushes the anchor driver towards the anchor in the loading site. In some implementations, the force generated by the driver spring relaxing causes drive head 136 to become locked onto anchor 140, e.g., the driver spring snaps the drive head into engagement with the anchor.

In some implementations, e.g., for implementations in which driver spring 128 is not present, the operator pushes anchor driver 130 distally towards loading site 129, such that the drive head engages with, and locks to, the anchor disposed within the loading site.

FIGS. 5A-C show drive head 136 engaged with the anchor that was moved into loading site 129 in FIGS. 4A-C, and the anchor having been advanced out of the loading site and into (e.g., through) aperture 123a, e.g., into catheter 160. Although anchor 140 typically exits loading site 129 while still housed within cartridge 150, as the anchor advances through aperture 123a cartridge 150 is stripped off of anchor 140, e.g., by the cartridge being prevented from passing through aperture 123a. Thus, in the example shown, cartridge 150 remains within housing 120, threaded around rod 134 (FIG. 5A).

In some implementations, the force applied by driver spring 128 pushing drive head 136 towards anchor 140 disposed in loading site 129 is sufficient to cause cuff 126 to push cartridge 150 out of the loading site through opening 129a. In some implementations, driver spring 128 also pushes anchor 140 out of cartridge 150 and through aperture 123a.

In some implementations, even if driver spring 128 generates sufficient force to lock drive head 136 to anchor 140 disposed in loading site 129, system 100 can be configured such that the human operator is required to manually push anchor driver 130 distally in order to advance anchor 140 (e.g., along with cartridge 150) out of loading site 129 and/or to advance anchor 140 through aperture 123a (e.g., without its cartridge).

In this state, anchor 140 can then be transluminally advanced through catheter 160 towards the heart (e.g., via the operator pushing anchor driver 130 distally), and anchored to tissue of the heart (FIGS. 6A-B) (e.g., tissue of a heart valve). Note that this advancement and anchoring can occur while cartridge 150 remains threaded on rod 134 of driver 130.

In some implementations, once the anchor is anchored to the tissue, anchor-release trigger 138 can be actuated to release the anchor from drive head 136, such that the anchor driver 130 can then be withdrawn proximally out of the heart and the subject, in order to anchor the next anchor of the series to the tissue (FIGS. 7A-C).

As described hereinabove, in some implementations, withdrawing anchor driver 130 proximally out of loading site 129 (e.g., in order to load the next anchor of the series into the loading site), allows the cartridge 150 that housed the previous anchor to become unthreaded from rod 134, and remain behind within receptacle 152 (e.g., falling into the receptacle and/or away from aperture 123a). This is most clearly shown in FIG. 7A.

In some implementations, the anchor (e.g., the second anchor of the series) that is now disposed in loading site 129 is then engaged by driver 130 and advanced out of the loading site and transluminally towards the heart, e.g., as described hereinabove.

In some implementations, this iterative process of withdrawing anchor driver 130 from loading site 129 such that the next anchor of the series is urged into the loading site, then advancing the anchor driver towards that anchor in the loading site, and then using the anchor driver to advance that anchor towards the heart, can be repeated for all the anchors housed within spiral channel 122. In some implementations, the operator can repeat this iterative process only until a desired number of anchors have been used.

FIG. 8 shows a back-view of anchor-storage device 110, with part of backplate 123 cut away to show multiple cartridges that have fallen into receptacle 152 following the use of the anchors that these cartridges previously housed.

In some implementations, once the anchoring process is finished, anchor driver 130 and anchor-storage device 110 (e.g., delivery assembly 102), can be withdrawn, leaving handle portion 104 and/or catheter 160 in place, e.g., for a subsequent step of the medical procedure. In some implementations, the entirety of system 100 can be withdrawn from the subject as a single entity.

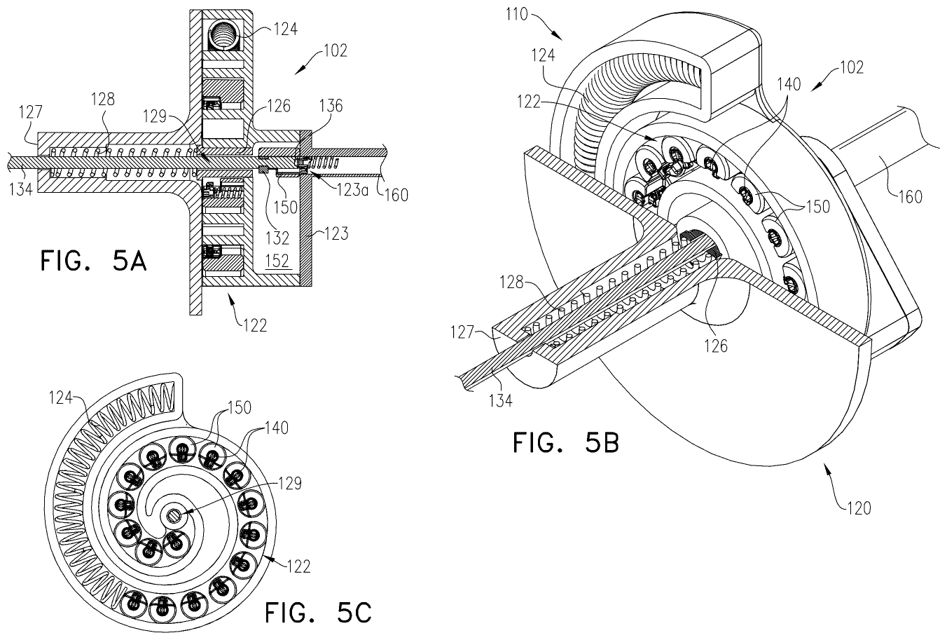

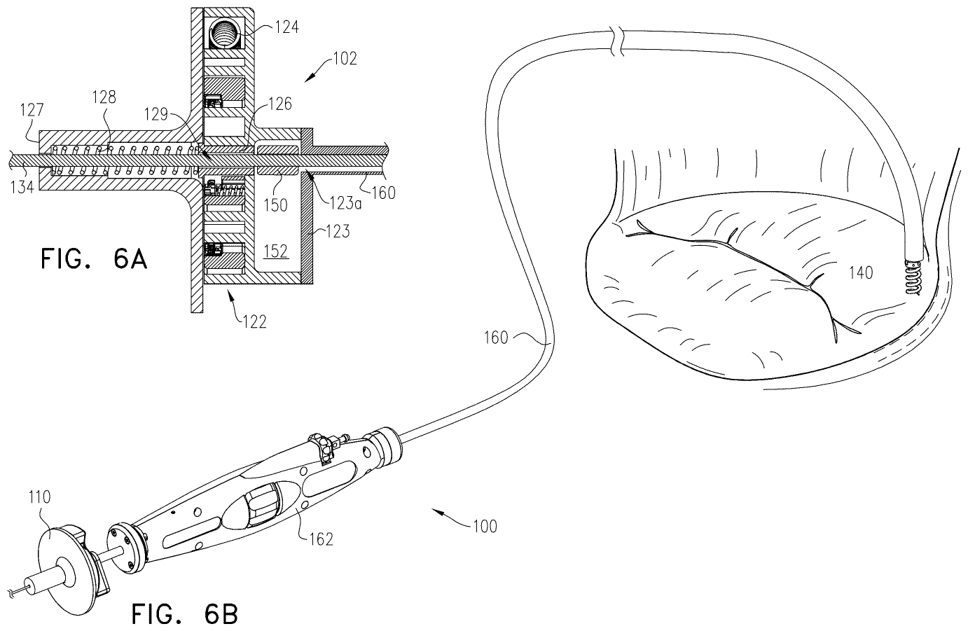

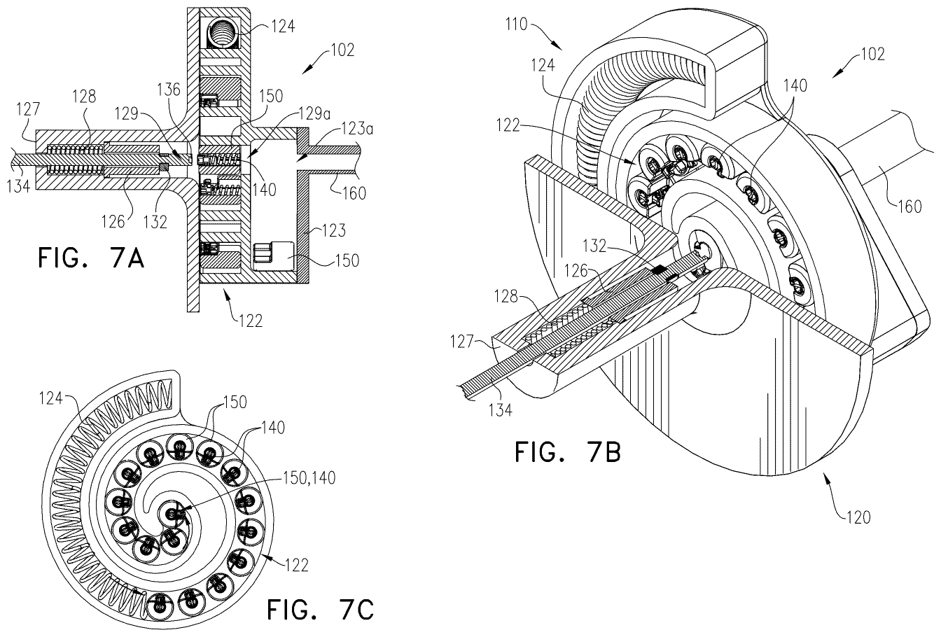

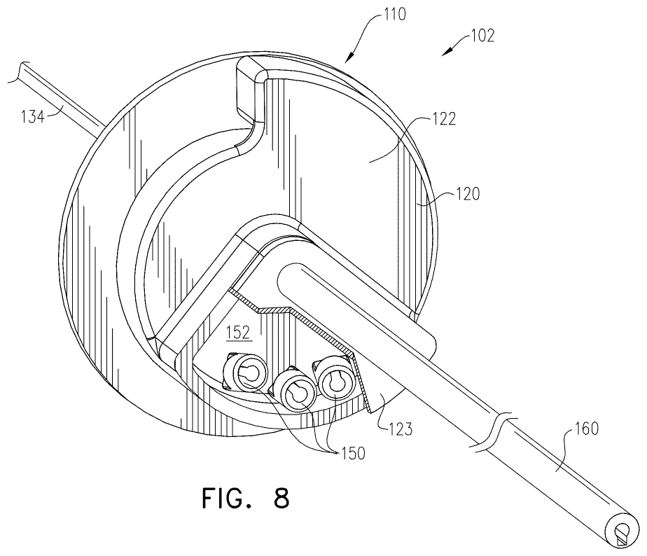

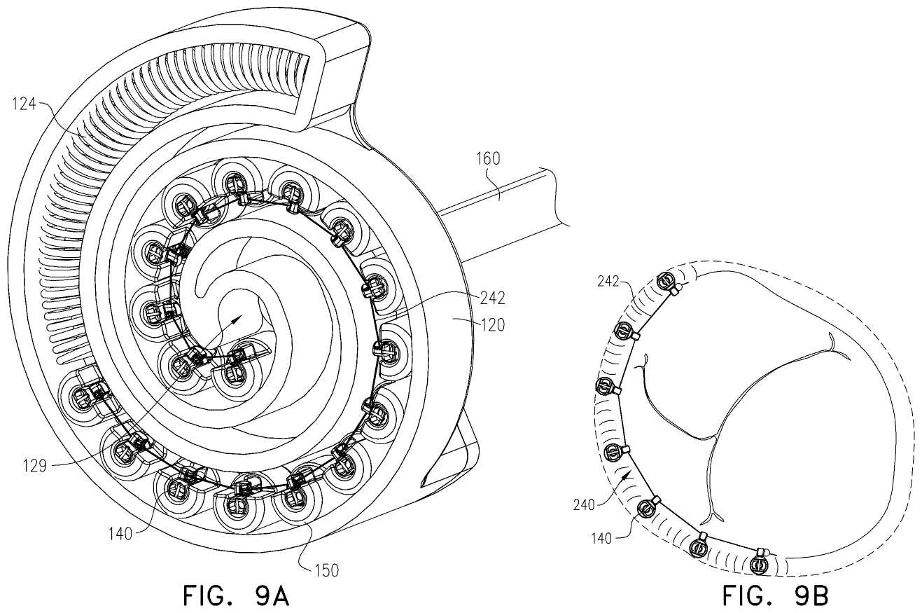

Reference is now made to FIGS. 9A-B, which are a schematic illustration of an example implant 240, and of an example delivery assembly 102 in use with the implant, in accordance with some implementations.

In some implementations, implant 240 comprises anchors 140, and a tether 242 that extends between the anchors (e.g., is threaded through an eyelet of the head of each of the anchors). In some implementations, delivery assembly 102 is adapted to anchor implant 240 to tissue of the heart while the tether extends between the anchors.

In some implementations, implant 240 can be an annuloplasty implant, and once the anchors have been anchored to the tissue, tether 242 can be tensioned in order to contract tissue 10 of the heart (FIG. 9B). In some implementations, prior to the anchors having been implanted within the heart, tether 242 extends along the anchors within spiral channel 122, e.g., as shown in FIG. 9A.

In some implementations, tether 242 can be advanced through loading site 129 and distally towards the heart. As shown, cartridges 150 can be shaped to accommodate tether 242 passing between anchors 140, e.g., at a proximal end of each cartridge one side of the cartridge can be cut away.

Reference is again made to FIGS. 1-9B. It is to be noted that delivery assembly 102 can be advantageously configured such that neither drive head 136 nor anchors 140 become exposed out of the delivery assembly during use, and do not require manual handling, e.g., thereby maintaining the anchors and their driver 130 in a sterile environment and reducing manual dexterity required for engaging the anchor driver with each anchor. For example, front plate 121 can be dimensioned to obstruct drive head 136 from being withdrawn proximally out of anchor-storage device 110 by defining an aperture 121a that allows rod 134, but not the drive head and/or protrusion 132, to be withdrawn proximally out of the aperture.

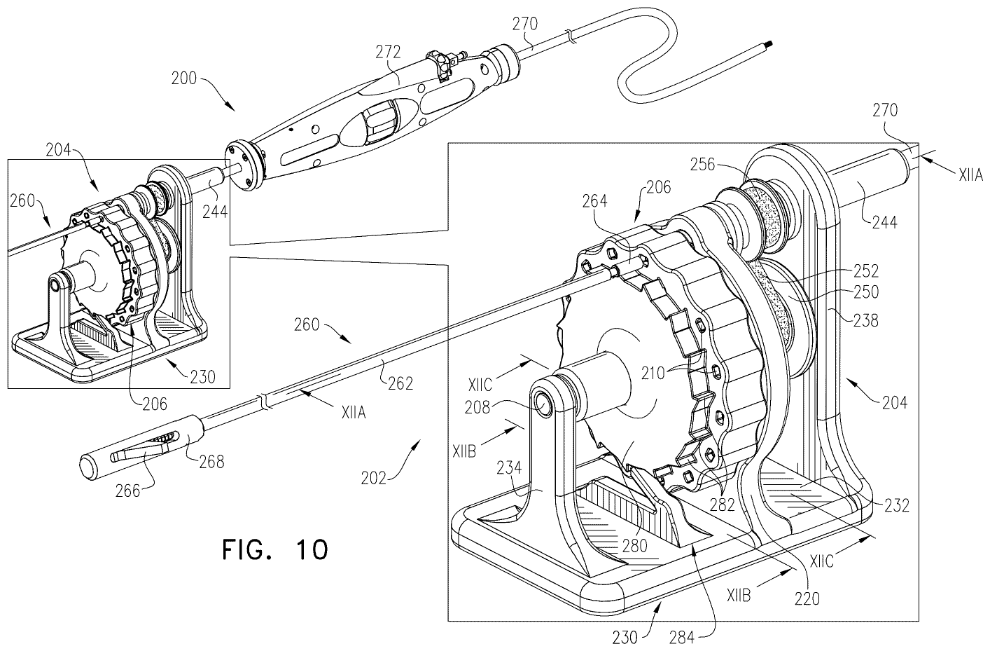

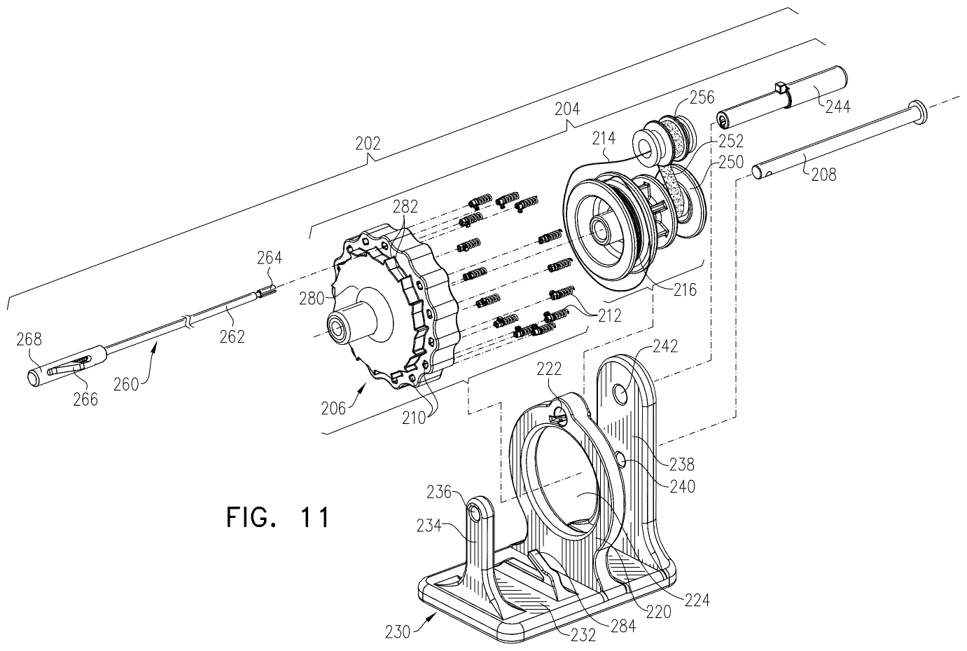

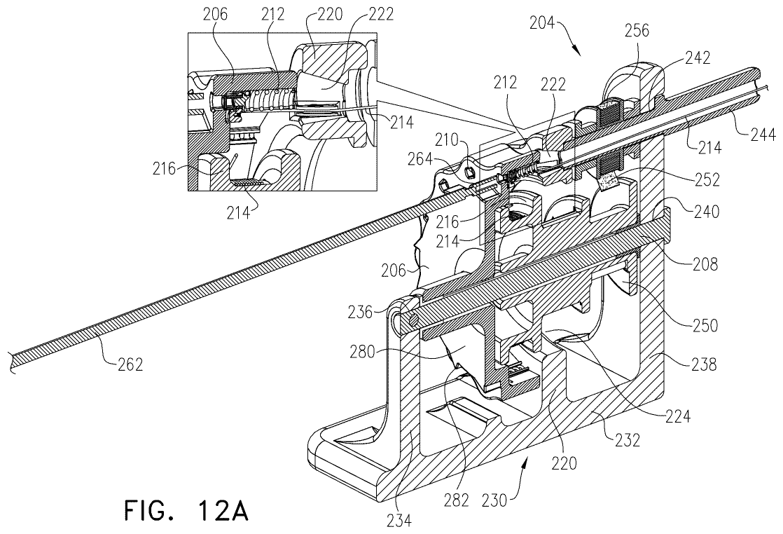

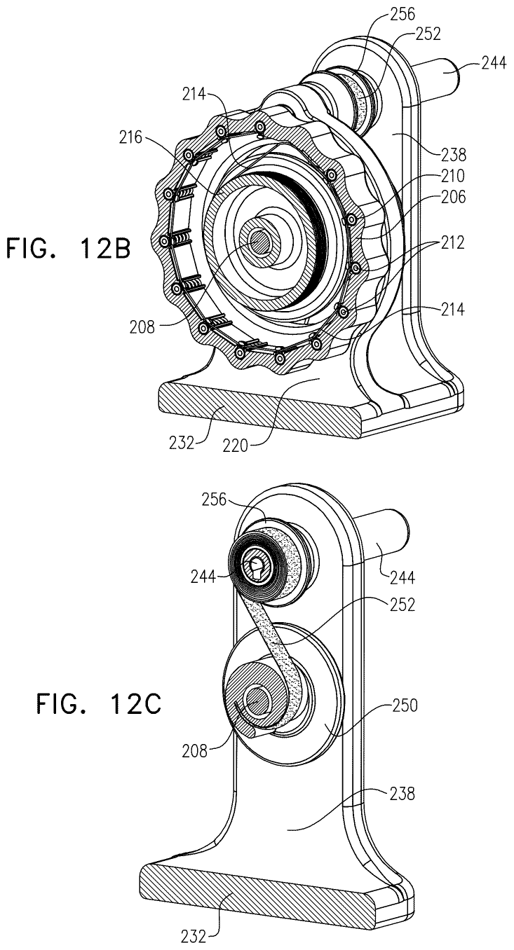

Reference is now made to FIG. 10, which is a schematic perspective illustration of an anchor-delivery system 200 according to some implementations, to FIG. 11, which is an exploded view illustration of an anchor-delivery assembly 202 forming part of anchor-delivery system 200, according to some implementations, and to FIGS. 12A, 12B, and 12C, which are sectional illustrations of the anchor-delivery assembly 202 according to some implementations.

In some implementations, anchor-storage device 204 differs from anchor-storage device 110 at least in that, rather than anchors being moved through the storage device into alignment for use, the storage device itself is rotated. In system 200, the anchors are disposed circumferentially around a housing 206 of the system, the housing being rotatable in order to iteratively bring each anchor into alignment for use.

Anchor-delivery assembly 202 includes an anchor-storage device 204, which comprises a housing 206, which is rotatable about an axis 208. Housing 206 defines a plurality of anchor-storage compartments 210, arranged around axis 208. Each of anchor-storage compartments 210 houses a tissue anchor 212 of a series of such tissue anchors, where each of tissue anchors 212 in the series is threaded onto a tether 214. Part of tether 214 is wound around a spool 216, which is also rotatable about axis 208. Rotation of spool 216 around axis 208 is independent from rotation of housing 206 around the axis.

Tissue anchors 212 and tether 214 together form an implant, such as an annuloplasty implant, adapted to be implanted in the heart of a subject, as explained in further detail hereinbelow.

Anchor-delivery assembly 202 further includes a backplate 220 defining an aperture 222. Each anchor 212 in the series is advanceable distally out of housing 206, through aperture 222. In some implementations, backplate 220 is annular, and includes a central bore 224 adapted to house spool 216 therein.

In some implementations, anchor-delivery assembly 202 includes a base structure 230 including a base surface 232. A proximal support 234 extends upwardly from base surface 232, and includes an indentation or bore 236, engaged by a proximal end of axis 208. A distal support 238 extends upwardly from base surface 232 and includes an indentation or bore 240 engaged by a distal end of axis 208. Distal support 238 can further include a second aperture 242, vertically aligned with aperture 222.

In some implementations, backplate 220 is disposed between proximal support 234 and distal support 238. In some implementations, an anchor-delivery channel 244 is disposed between aperture 222 and second aperture 242.

Spool 216 is functionally associated with a first spring-spool 250, adapted to receive axis 208 therein. A spring 252, such as a leaf spring, is coupled to first spring-spool 250, and extends to a second spring-spool 256, mounted onto anchor-delivery channel 244. As explained in further detail hereinbelow, spring 252 is adapted to take up slack of (e.g. to maintain some tension on) tether 214 by causing rotation of spool 216, as necessary. In some implementations, rotation of spring 252 re-distributes the portion of the spring spooled around each of spring-spools 250 and 256.

Anchor-delivery assembly 202 further includes an anchor driver 260, slidably coupled to anchor storage device 204. Anchor driver 260 includes a rod 262, terminating, at a distal end thereof, in a drive head 264. In some implementations, anchor driver 260 includes an anchor-release mechanism, disposed at a proximal end of rod 262. For example, the anchor-release mechanism can include an anchor-release trigger 266 disposed in a driver handle 268 of anchor driver 260.

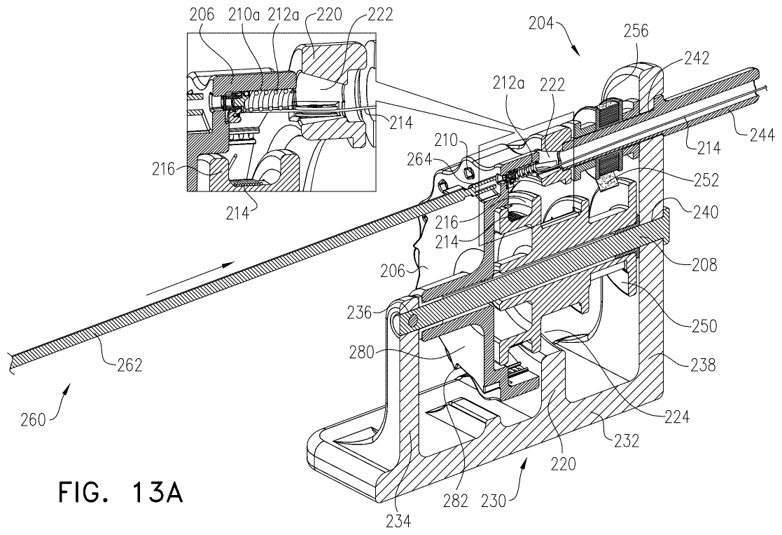

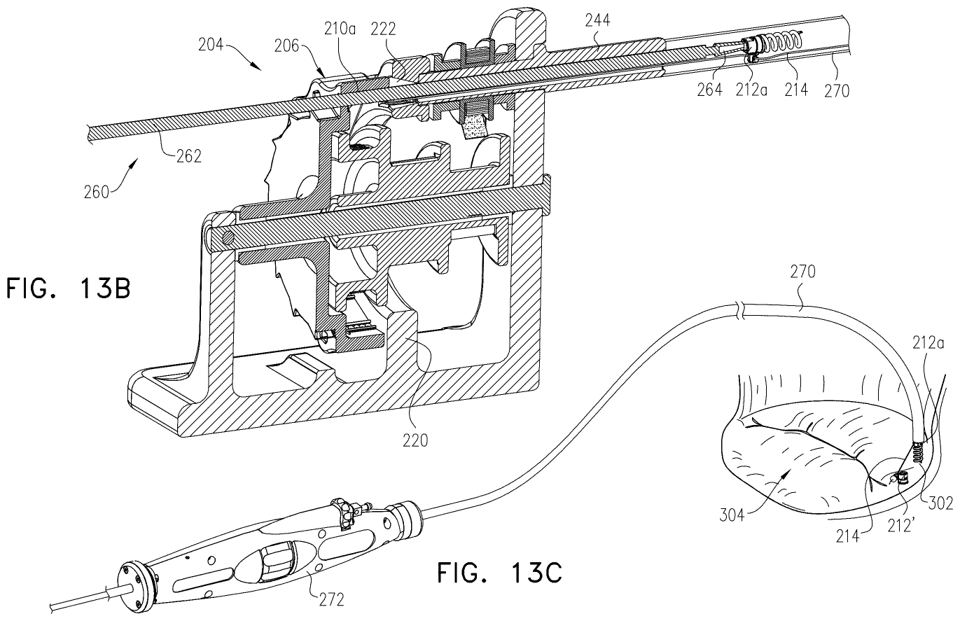

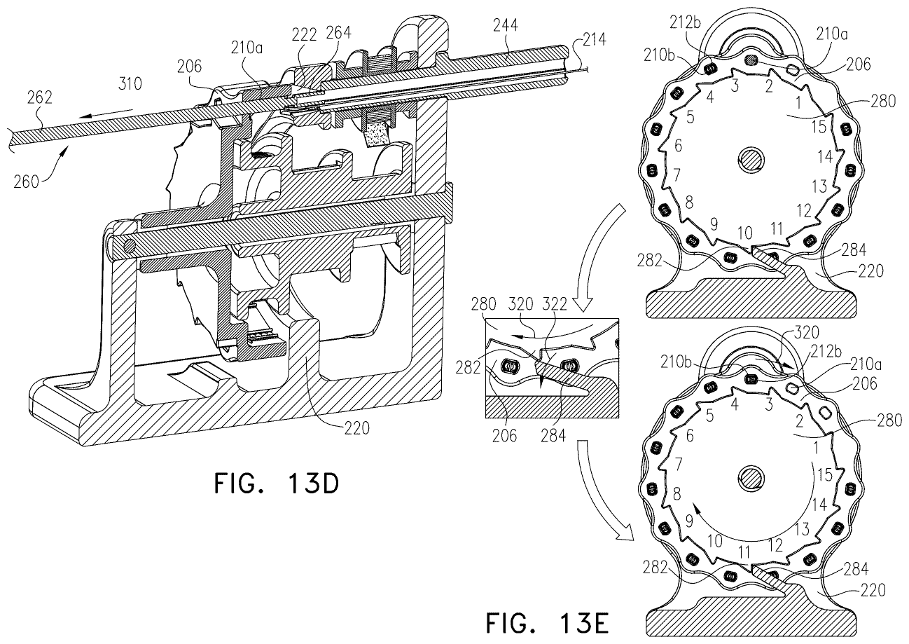

As explained in further detail hereinbelow with respect to FIGS. 13A to 13E, anchor driver 260 is extendable through aperture 222 to drive an anchor 212 distally out of one of anchor-storage compartments 210 of housing 206, aligned with the aperture, into anchor-delivery channel 244, and further out anchor-storage device 204 and into the heart of the subject. Once the anchor 212 is anchored to the heart of the subject, the anchor-release mechanism is adapted to release the anchor from the drive head 264. In some implementations, anchor driver 260 and/or its anchor-release mechanism can be as described, mutatis mutandis, in one or more of the following publications, each of which is incorporated herein by reference:
U.S. Patent Application Publication No. 2012/0022557 to Cabiri et al.
U.S. Patent Application Publication No. 2014/0309661 to Sheps et al.
U.S. Patent Application Publication No. 2015/0272734 to Sheps et al.
U.S. Patent Application Publication No. 2018/0049875 to Iflah et al.
U.S. Patent Application Publication No. 2022/0071620 to Brauon et al.
International Patent Application Publication No. WO 2022/064401 to Halabi et al.
International Patent Application No. PCT/IB2022/051099 to Shafigh et al.

In some implementations, anchor-delivery system further includes a catheter 270, coupled to anchor-storage device 204 and extending distally therefrom. For example, a lumen of catheter 270 can extend distally from anchor-delivery channel 244 of anchor-storage device 204. As explained in further detail hereinbelow, anchor driver 260 can delivery anchors 212 to the heart of the subject transluminally (e.g., transfemorally), via catheter 270.

In some implementations, a catheter handle 272 is positioned at a proximal part of catheter 270, the catheter handle being adapted to facilitate transluminal advancement of the catheter towards the heart, e.g., by steering a distal steering portion of the catheter via operational coupling (e.g., via pullwires) between the catheter handle and the distal steering portion. Nonlimiting examples of how this steering can be implemented include those described in the following publications, each of which is incorporated by reference:
U.S. Patent Application Publication No. 2014/0309661 to Sheps et al.
U.S. Patent Application Publication No. 2015/0272734 to Sheps et al.

In some implementations, and as shown in FIG. 10, anchor-storage device 204 can be positioned proximally to catheter handle 272, such as affixed to the proximal end of the catheter handle.

As explained in further detail hereinbelow, during implantation of anchors 212, housing 206 must be rotated relative to backplate 220, so as to align each of anchors 212 with aperture 222, for driving of the specific anchor into anchor-delivery channel 244, and further into catheter 270.

In some implementations, housing 206 is manually rotated relative to backplate 220. In some implementations, housing 206 is coupled to a ratcheting mechanism 280, defining ratcheting teeth 282. Ratcheting teeth 282 are functionally associated with an engagement arm 284 extending upwardly from base 232 of base structure 230. Ratcheting mechanism 280 together with engagement arm 284 enable measured rotation of housing 206 relative to backplate 220, to ensure that none of anchors 212 are missed during the implantation process.

In some implementations, housing 206 can be automatically rotated relative to backplate 220. In some implementations, housing 206 and anchor driver 260 can have a structure similar to that described hereinabove with respect to FIGS. 1 to 9B, such that the anchor driver is pulled proximally to compress a driver spring, which causes rotation of housing 206 prior to release of the anchor driver to engage the next anchor in the series. In some implementations, such an automated system can include components of the system described hereinabove with respect to FIGS. 1 to 9B, mutatis mutandis.

In some implementations, each anchor-storage compartment houses a cartridge or capsule (similar to cartridges 150 of FIG. 2), and anchor 212 is disposed within the cartridge. Characteristics of such cartridges are described hereinabove with respect to FIGS. 1 to 9B. In some implementations, when anchor driver 260 pushes an anchor 212 distally out of housing 206, the cartridge can be retained in the housing, or can be disposed about the rod of the anchor driver and then dropped into a suitable receptacle, as described hereinabove with respect to FIGS. 1 to 9B, mutatis mutandis.

Reference is now additionally made to FIGS. 13A, 13B, 13C, 13D, and 13E, which are schematic illustrations of at least some steps of a process of using the anchor-delivery assembly 202 for the delivery and implantation of tissue anchor 212 of a series of tissue anchors stored within anchor-storage device 204 of the anchor-delivery assembly.

As seen in FIG. 13A, a specific anchor-storage compartment 210a of housing 206, houses a specific anchor 212a, which is threaded onto tether 214. In the illustrated example, a previous anchor has already been implanted, such that tether 214 extends distally to anchor 212a, through aperture 222 and anchor-delivery channel 244.

Anchor-storage compartment 210a is aligned with aperture 222 of backplate 220 and with anchor-delivery channel 244. Anchor driver 260 is disengaged from housing 206, with drive head 264 being aligned with anchor-storage compartment 210a.

In FIG. 13B, drive head 264 of anchor driver 260 has engaged anchor 212a, and has pushed the anchor distally out of housing 206, via aperture 222 of backplate 220 and via anchor-delivery channel 244, into catheter 270. As shown, rod 262 of anchor driver 260 continues to extend through housing 206, aperture 222, and anchor-delivery channel 244, during this process.

While drive head 264 remains engaged with anchor 212a, rod 262 continues to transluminally advance the anchor to the heart of the subject, through catheter 270. Subsequently, while rod 262 remains extending through aperture 222 and channel 244, anchor driver 260 drives anchor 212a into tissue of the heart of the subject, as illustrated in FIG. 13C. For example, anchor 212a can be driven into annulus 302 of the mitral valve 304 of the subject. As seen, another anchor 212′ has already been driven into the annulus 302 and is connected to specific anchor 212a by tether 214.

Turning to FIG. 13D, it is seen that following installation of anchor 212a, drive head 264 is disengaged from anchor 212a, for example by operation of the anchor-release mechanism. Anchor driver 260, and particularly rod 262, is then drawn in a proximal direction, as indicated by arrow 310, until drive head 264 is removed from anchor-storage compartment 210a and/or from housing 206.

Following withdrawal of anchor driver 260, housing 206 is rotated relative to backplate 220, to bring another anchor 212b disposed in another anchor-storage compartment 210b to be in alignment with aperture 222. In this position, the anchor may be considered to be at a loading site of anchor-storage device 204. FIG. 13E illustrates rotation of housing 206 from a first position, in which anchor-storage compartment 210a is aligned with aperture 222, in the direction of arrows 320, to bring anchor-storage compartment 210b to be aligned with aperture. During such rotation, engagement arm 284 is pushed downward, in the direction of arrow 322, by ratcheting teeth 282, and re-engages the next tooth in the mechanism, thereby resulting in measured rotation of the housing and ensuring that no anchor is skipped.

In some implementations, this rotation of housing 206 is performed manually by the operator. In some implementations, this rotation is semi-or fully-automatic. For example, the rotation may be effected by a spring—e.g. by withdrawal of anchor driver 260 to compress a release spring.

It is to be appreciated that housing 206 is rotatable relative to backplate 220 when anchor driver 260 is disengaged from aperture 222, or fully disengaged from the housing. Additionally, the housing is not rotatable relative to backplate 220 when anchor driver 260 extends through the aperture.

It is to be appreciated that the process shown in FIGS. 13A to 13E can be repeated until all anchors 212 in the series have been advanced into the heart of the subject, for example to form the implanted structure shown in FIG. 9B.

It should be understood that the use of “and/or” is defined inclusively such that the term “a and/or b” should be read to include the sets: “a and b,” “a or b,” “a,” “b.”

Example Implementations (some non-limiting examples of the concepts herein are recited below):
Example 1. A system, comprising: (A) a series of anchors; (B) an anchor-storage device, comprising: (i) a spring; and/or (ii) a housing, shaped to define: (a) a loading site, and/or (b) a spiral channel that spirals inward to the loading site, the series of anchors arranged along the spiral channel, the spring configured to urge the series of anchors along the spiral channel toward the loading site; and/or (C) an anchor driver, slidably coupled to the anchor-storage device, and comprising a rod extendable through the loading site in a manner that obstructs access of the series of anchors to the loading site from the spiral channel.
Example 2. The system according to example 1, wherein the system comprises an implant, the implant comprising the series of anchors, and a tether that extends between the anchors of the series.
Example 3. The system according to any one of examples 1-2, wherein the spring lies along a portion of the channel.
Example 4. The system according to any one of examples 1-3, wherein: (a) the anchor driver defines a drive head at a distal end of the rod, and/or (b) for each anchor of the series, while the anchor is disposed in the loading site, the anchor is configured to be engaged by the drive head.
Example 5. The system according to example 4, wherein the anchor driver is configured: (a) while the drive head remains engaged with the anchor, to transluminally advance the anchor to a heart of a subject by advancing the rod distally through the loading site, and/or (b) while the rod remains extended through the loading site, to anchor the anchor to tissue of the heart.
Example 6. The system according to example 5, wherein the system further comprises an anchor-release mechanism at a proximal end of the system, adapted to release the anchor from the drive head once the anchor is anchored to the tissue.
Example 7. The system according to one of examples 1-6, wherein the urging of the series of anchors by the spring automatically moves an anchor of the series into the loading site upon withdrawal of the rod from the loading site.
Example 8. The system according to example 7, wherein the spring is disposed within the spiral channel, and is configured to automatically expand within the channel upon withdrawal of the rod from the loading site.
Example 9. The system according to example 7, wherein: (a) the anchor driver defines a drive head at a distal end of the rod, and/or (b) the housing defines a proximal stop that obstructs the drive head from exiting the anchor-storage device proximally.
Example 10. The system according to example 9, wherein the rod is slidable through the proximal stop.
Example 11. The system according to example 9, wherein: (a) the system further comprises a driver spring disposed within the housing, and/or (b) the anchor-storage device is configured such that withdrawing the anchor driver proximally out of the loading site compresses the driver spring against the proximal stop.
Example 12. The system according to example 11, wherein the driver spring is a helical compression spring.
Example 13. The system according to example 11, wherein: (a) the anchor driver further defines a lateral protrusion at a distal portion of the rod, and/or (b) the lateral protrusion is dimensioned such that withdrawing the anchor driver proximally out of the loading site causes the lateral protrusion to compress the driver spring against the proximal stop.
Example 14. The system according to example 13, wherein the anchor driver comprises a ring that is mounted on the distal portion of the rod, and that defines the lateral protrusion.
Example 15. The system according to example 13, wherein the anchor-storage device further comprises a cuff threaded onto the rod, and reversibly positionable within the loading site.
Example 16. The system according to example 15, wherein the lateral protrusion and the cuff are shaped and positioned such that withdrawing the anchor driver proximally out of the loading site causes the lateral protrusion to force the cuff proximally to compress the driver spring against the proximal stop.
Example 17. The system according to example 16, wherein the anchor driver is extendable through the loading site in a manner that positions the cuff within the loading site in a manner that obstructs access of the series of anchors to the loading site from the spiral channel.
Example 18. The system according to example 11, wherein the driver spring is configured to push the drive head to the anchor at the loading site.
Example 19. The system according to example 18, wherein the drive head is configured to automatically lock onto the anchor upon being pushed to the anchor by the driver spring.
Example 20. The system according to one of examples 1-19, wherein the housing defines an opening in communication with the loading site, configured to allow the anchor driver to advance each anchor distally out of the loading site.
Example 21. The system according to example 20, wherein the spiral channel circumscribes an axis, and wherein the opening is configured to allow the anchor driver to advance each anchor out of the loading site distally along the axis.
Example 22. The system according to example 21, wherein the spring arcs around axis.
Example 23. The system according to example 20, wherein: (a) the anchor driver has a drive head at distal end of the rod, and/or (b) for each anchor: (i) the drive head is configured to engage the anchor, and/or (ii) the anchor driver is configured to transluminally anchor the anchor to tissue of a heart of a subject by, while the rod remains extended through the loading site, advancing the drive head and the anchor distally through the opening, out of the anchor-storage device, and transluminally to the heart.
Example 24. The system according to example 23, further comprising a series of cartridges, each cartridge of the series housing a corresponding anchor of the series, and being arranged along the spiral channel.
Example 25. The system according to example 24, wherein, for each cartridge of the series, the housing is configured to retain the cartridge while the anchor driver advances the corresponding anchor distally out of the anchor-storage device.
Example 26. The system according to example 25, wherein the housing defines a receptacle that is in communication with the loading site via the opening, and that is dimensioned to retain each cartridge of the series within the receptacle.
Example 27. The system according to example 26, wherein the receptacle is positioned distally to the spiral channel.
Example 28. The system according to example 26, wherein the receptacle defines a backplate that defines an aperture that, for each anchor of the series, is dimensioned to allow the anchor, but not the corresponding cartridge, to pass distally out of the housing.
Example 29. The system according to example 28, wherein each cartridge of the series is shaped to remain threaded on the rod, within the receptacle, while the anchor driver advances the corresponding anchor distally out of the anchor-storage device.
Example 30. The system according to example 29, wherein each cartridge of the series is shaped such that withdrawing the anchor driver proximally out of the receptacle unthreads the cartridge from the rod such that the cartridge is retained within the receptacle.
Example 31. The system according to example 23, wherein: (a) the system further comprises a catheter, coupled to the housing and extending distally away from the housing, and/or (b) the anchor driver is configured to transluminally anchor the anchor to the tissue of the heart by, while the rod remains extended through the loading site, advancing the drive head and the anchor distally through the opening, out of the anchor-storage device, and through the catheter to the heart.
Example 32. The system according to example 31, wherein the system further comprises a catheter control handle, adapted to facilitate transluminal steering of the catheter to the heart.
Example 33. The system according to example 32, wherein the catheter control handle is positioned axially between the anchor-storage device and the catheter.
Example 34. A method for use with a subject, the method comprising: (a) advancing a first anchor distally out of a loading site defined by a housing of an anchor- storage device, and transluminally to a tissue of the subject, by sliding, distally through the loading site, an anchor driver engaged with the anchor, the housing defining a spiral channel that spirals inward to the loading site; (b) subsequently, within the subject, disengaging the anchor driver from the anchor; and/or (c) subsequently, withdrawing the anchor driver proximally through and out of the loading site such that the anchor-storage device automatically advances a second anchor from the spiral channel into the loading site.
Example 35. The method according to example 34, wherein: (a) the anchor driver includes a drive head, (b) sliding the anchor driver engaged with the anchor comprises sliding the anchor driver while the drive head is engaged with the anchor, and/or (c) withdrawing the anchor driver proximally through and out of the loading site comprises withdrawing the anchor driver proximally out of the loading site without withdrawing the drive head from the anchor-storage device.
Example 36. The method according to any one of examples 34-35, wherein transluminally advancing the first anchor to the tissue comprises transluminally advancing the first anchor to the tissue through a catheter.
Example 37. The method according to example 36, wherein the method further comprises actively steering the catheter towards the tissue via vasculature of the subject.
Example 38. The method according to any one of examples 34-37, wherein withdrawing the anchor driver proximally through and out of the loading site comprises withdrawing the anchor driver proximally out of the loading site such that a driver spring becomes compressed within the anchor-storage device.
Example 39. The method according to example 38, wherein the method further comprises, subsequently to the withdrawing, releasing the anchor driver such that the driver spring pushes the anchor driver towards the second anchor in the loading site.
Example 40. The method according to example 39, wherein releasing the anchor driver such that the driver spring pushes the anchor driver towards the second anchor in the loading site comprises releasing the anchor driver such that the anchor driver locks onto the second anchor.
Example 41. The method according to any one of examples 34-40, wherein the first anchor is housed in a cartridge, and wherein transluminally advancing the first anchor to the tissue comprises advancing the first anchor out of the cartridge and the anchor-storage device while the cartridge remains within the anchor-storage device.
Example 42. The method according to example 41, wherein transluminally advancing the first anchor to the tissue comprises transluminally advancing the first anchor to the tissue by sliding the anchor driver through the cartridge.
Example 43. The method according to example 41, wherein advancing the first anchor out of the cartridge and the anchor-storage device comprises advancing the first anchor out of the cartridge and the anchor-storage device while the cartridge remains within the anchor-storage device, threaded on the anchor driver.
Example 44. The method according to example 43, further comprising, subsequently to disengaging the anchor driver from the anchor, unthreading the anchor driver from the cartridge by withdrawing the anchor driver proximally out of the cartridge.
Example 45. An apparatus, comprising: (a) a tether; (b) a series of anchors, threaded onto the tether; and/or (c) an anchor-storage device, comprising: (i) a housing, rotatable about an axis, and comprising a plurality of anchor storage compartments arranged around the axis, each of the anchor storage compartments housing a respective anchor of the series of anchors; (ii) a backplate defining an aperture through which each anchor of the series is advanceable distally out of the housing; and/or (iii) a spool having part of the tether wound therearound, wherein the housing is rotatable relative to the backplate, in a manner that enables alignment of each of the anchor storage compartments, in sequence, to become aligned with the aperture.
Example 46. The apparatus according to example 45, wherein the apparatus comprises an implant, the implant comprising the tether and the series of anchors.
Example 47. The apparatus according to any one of examples 45-46, further comprising an anchor driver, slidably coupled to the anchor-storage device, and comprising a rod extendable through the aperture to drive an anchor of the series of anchors, disposed in one of said plurality of anchor storage compartments aligned with the aperture, distally out of the anchor-storage device.
Example 48. The apparatus according to example 47 wherein the housing is rotatable relative to the backplate when the anchor driver is disengaged from the aperture.
Example 49. The apparatus according to any one of examples 47-48 wherein the housing is not rotatable relative to the backplate when the anchor driver extends through the aperture.
Example 50. The apparatus according to any one of examples 45-49, wherein the housing is manually rotatable relative to the backplate.
Example 51. The apparatus according to example 50, wherein the anchor-storage device further includes an engagement arm, and an exterior surface of the housing includes a ratchet mechanism adapted to be engaged by the engagement arm, to enable measured rotation of the housing relative to the backplate.
Example 52. The apparatus according to any one of examples 47-49, wherein the housing is automatically rotatable relative to the backplate.
Example 53. The apparatus according to example 52, wherein: (a) the anchor driver defines a drive head at a distal end of the rod, and/or (b) the housing defines a proximal stop that obstructs the drive head from exiting the anchor-storage device proximally.
Example 54. The apparatus according to example 53, wherein the rod is slidable through the proximal stop.
Example 55. The apparatus according to any one of examples 53-54, wherein: (a) the apparatus further comprises a driver spring disposed within the housing, and/or (b) the anchor-storage device is configured such that withdrawing the anchor driver proximally out of the aperture in the backplate compresses the driver spring against the proximal stop.
Example 56. The apparatus according to example 55, wherein the driver spring is a helical compression spring.
Example 57. The apparatus according to any one of examples 55-56, wherein: (a) the anchor driver further defines a lateral protrusion at a distal portion of the rod, and/or (b) the lateral protrusion is dimensioned such that withdrawing the anchor driver proximally out of the aperture in the backplate causes the lateral protrusion to compress the driver spring against the proximal stop.
Example 58. The apparatus according to any one of examples 55-57, wherein the anchor driver comprises a ring that is mounted on the distal portion of the rod, and that defines the lateral protrusion.
Example 59. The apparatus according to any one of examples 55-58, wherein the anchor-storage device further comprises a cuff threaded onto the rod, and reversibly positionable within the loading site.
Example 60. The apparatus according to example 59, wherein the lateral protrusion and the cuff are shaped and positioned such that withdrawing the anchor driver proximally out of the loading site causes the lateral protrusion to force the cuff proximally to compress the driver spring against the proximal stop.
Example 61. The apparatus according to any one of examples 55-60, wherein the driver spring is configured to push the drive head to the anchor aligned with the aperture.
Example 62. The apparatus according to any one of examples 55-61, wherein the drive head is configured to automatically lock onto the anchor upon being pushed to the anchor by the driver spring.
Example 63. The apparatus according to any one of examples 45-62, wherein the spool is rotatable relative to the backplate.
Example 64. The apparatus according to example 63, wherein the spool is rotatable independently of rotation of the housing.
Example 65. The apparatus according to any one of examples 63-64, wherein rotation of the spool tensions the tether during delivery of one or more of the series of tissue anchors distally out of the housing.
Example 66. The apparatus according to example 65, wherein the spool is functionally associated with a spring, the spring configured rotate the spool thereby to tension the tether.
Example 67. The apparatus according to any one of examples 45-66, wherein: (a) the anchor driver defines a drive head at a distal end of the rod, and/or (b) for each anchor of the series, while the anchor is aligned with the aperture in the backplate, the anchor is configured to be engaged by the drive head.
Example 68. The apparatus according to example 67, wherein the anchor driver is configured: (a) while the drive head remains engaged with the anchor, to transluminally advance the anchor to a heart of a subject by advancing the rod distally through the aperture in the backplate, and/or (b) while the rod remains extended through the aperture, to anchor the anchor to tissue of the heart.
Example 69. The apparatus according to example 68, wherein the apparatus further comprises an anchor-release mechanism at a proximal end of the apparatus, adapted to release the anchor from the drive head once the anchor is anchored to the tissue.
Example 70. The apparatus according to any one of examples 45-69, further comprising a series of cartridges, each cartridge of the series housing a corresponding anchor of the series, the cartridges being arranged around the axis of the housing.
Example 71. The apparatus according to any one of examples 45-70, wherein: (a) the apparatus further comprises a catheter, coupled to the housing and extending distally away from the housing, and/or (b) the anchor driver is configured to transluminally anchor the anchor to the tissue of the heart by, while the rod remains extended through the aperture in the backplate, advancing the drive head and the anchor distally out of the anchor-storage device, and through the catheter to the heart.
Example 72. The apparatus according to example 71, wherein the apparatus further comprises a catheter control handle, adapted to facilitate transluminal steering of the catheter to the heart.
Example 73. A method for use with a subject, the method comprising: (a) obtaining an anchor storage device including a housing rotatable about an axis, the housing comprising a plurality of anchor storage compartments arranged around the axis, each of the anchor storage compartments housing a respective anchor of a series of anchors threaded onto a tether, the anchor storage device further including a backplate defining an aperture; (b) advancing a first anchor of the series of anchors distally out of one of the anchor storage compartment in the housing, via the aperture in the backplate, and transluminally to a tissue of the subject, by sliding, distally through the aperture, an anchor driver engaged with the anchor; (c) subsequently, within the subject, disengaging the anchor driver from the anchor and withdrawing the anchor driver proximally through and out of the aperture; and/or (d) subsequently, rotating the housing about the axis relative to the backplate to align a second anchor of the series with the aperture in the backplate.
Example 74. The method according to example 73, wherein the rotating comprises manually rotating the housing about the axis.
Example 75. The method according to example 74, wherein: (a) the anchor driver includes a drive head, (b) sliding the anchor driver engaged with the anchor comprises sliding the anchor driver while the drive head is engaged with the anchor, and/or (c) withdrawing the anchor driver proximally through and out of the aperture comprises withdrawing the anchor driver proximally out of the aperture while withdrawing the drive head from the anchor-storage device.
Example 76. The method according to example 73, wherein the rotating comprises automatically rotating the housing about the axis.
Example 77. The method according to example 76, wherein: (a) the anchor driver includes a drive head, (b) sliding the anchor driver engaged with the anchor comprises sliding the anchor driver while the drive head is engaged with the anchor, and/or (c) withdrawing the anchor driver proximally through and out of the aperture comprises withdrawing the anchor driver proximally out of the aperture without withdrawing the drive head from the anchor-storage device.
Example 78. The method according to any one of examples 76-77, wherein withdrawing the anchor driver proximally through and out of the loading site comprises withdrawing the anchor driver proximally out of the loading site such that a driver spring becomes compressed within the anchor-storage device.
Example 79. The method according to example 78, wherein the automatically rotating comprises, subsequently to the withdrawing, releasing the anchor driver such that the driver spring rotates the housing to align the second anchor with the aperture.
Example 80. The method according to example 79, wherein releasing the anchor driver comprises releasing the anchor driver such that the anchor driver locks onto the second anchor aligned with the aperture.
Example 81. The method according to any one of examples 73-80, wherein transluminally advancing the first anchor to the tissue comprises transluminally advancing the first anchor to the tissue through a catheter.
Example 82. The method according to example 81, wherein the method further comprises actively steering the catheter towards the tissue via vasculature of the subject.
Example 83. The method according to any one of examples 73-82, wherein the first anchor is housed in a cartridge, and wherein transluminally advancing the first anchor to the tissue comprises advancing the first anchor out of the cartridge and the anchor-storage device while the cartridge remains within the anchor-storage device.
Example 84. The method according to example 83, wherein transluminally advancing the first anchor to the tissue comprises transluminally advancing the first anchor to the tissue by sliding the anchor driver through the cartridge.
Example 85. The method according to any one of examples 83-84, wherein advancing the first anchor out of the cartridge and the anchor-storage device comprises advancing the first anchor out of the cartridge and the anchor-storage device while the cartridge remains within the anchor-storage device, threaded on the anchor driver.
Example 86. The method according to example 85, further comprising, subsequently to disengaging the anchor driver from the anchor, unthreading the anchor driver from the cartridge by withdrawing the anchor driver proximally out of the cartridge.
Example 87. The method according to any one of examples 73-86, further comprising, repeating the steps of advancing, disengaging, and rotating, for each anchor in the series of anchors.

Any of the various systems, assemblies, devices, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, apparatus, etc. (e.g., with heat, radiation, ethylene oxide, hydrogen peroxide, etc.).

Furthermore, although reference has been made to tissue anchors throughout the application, it is to be understood that the methods and systems described herein of housing and handling tissue anchors could be used for other implantable and/or anchorable elements other than tissue anchors, e.g., neurostimulators, leadless pacemakers, darts, hooks, staples, sutures, etc. In some implementations, cartridges such as cartridges 150 may or may not be used to house such elements within the storage device.

The treatment techniques, methods, operations, steps, etc. described or suggested herein or in the references incorporated herein can be performed on a living subject (e.g., human, other animal, etc.) or on a non-living simulation, such as a cadaver, cadaver heart, simulator, imaginary person, etc. When performed on a simulation, the body parts, e.g., heart, tissue, valve, etc., can optionally be referred to as “simulated” (e.g., simulated heart, simulated tissue, simulated valve, etc.) and can comprise, for example, computerized and/or physical representations of body parts, tissue, etc.

What is claimed is:

1. A system, comprising:
a series of implantable anchors;
an anchor-storage device, comprising:
a spring; and
a housing, shaped to define:
a loading site, and
a spiral channel that spirals inward to the loading site, the series of anchors arranged along the spiral channel, the spring configured to urge the series of anchors along the spiral channel toward the loading site; and
an anchor driver:
slidably coupled to the anchor-storage device,
comprising a rod,
defining a drive head at a distal end of the rod,
for each anchor of the series, while the anchor is disposed in the loading site, configured to engage the anchor using the drive head,
while the drive head remains engaged with the anchor, configured to transluminally advance the anchor to a cardiovascular tissue of a subject by advancing the rod distally through the loading site, and
while the rod remains disposed through the loading site, configured to anchor the anchor to the tissue.

2. The system according to claim 1, wherein the system comprises an implant, the implant comprising:
the series of anchor, and
a tether that extends between the anchors of the series.

3. The system according to claim 2, wherein, for each anchor of the series, the tether is threaded through an eyelet defined by the anchor.

4. The system according to claim 1, wherein the spring lies along a portion of the channel.

5. The system according to claim 1, wherein the rod is extendable through the loading site in a manner that obstructs access of the series of anchors to the loading site from the spiral channel.

6. The system according to claim 5, wherein the system further comprises an anchor-release mechanism at a proximal end of the system, adapted to release the anchor from the drive head once the anchor is anchored to the tissue.

7. The system according to claim 1, wherein the urging of the series of anchors by the spring automatically moves an anchor of the series into the loading site upon withdrawal of the rod from the loading site.

8. The system according to claim 7, wherein the housing defines a proximal stop that obstructs the drive head from exiting the anchor-storage device proximally.

9. The system according to claim 8, wherein the rod is slidable through the proximal stop.

10. The system according to claim 8, wherein:
the system further comprises a driver spring disposed within the housing, and
the anchor-storage device is configured such that withdrawing the anchor driver proximally out of the loading site compresses the driver spring against the proximal stop.

11. The system according to claim 10, wherein the driver spring is configured to push the drive head to the anchor at the loading site.

12. The system according to claim 11, wherein the drive head is configured to automatically lock onto the anchor upon being pushed to the anchor by the driver spring.

13. The system according to claim 1, wherein the housing defines an opening in communication with the loading site, configured to allow the anchor driver to advance each anchor distally out of the loading site.

14. The system according to claim 13, wherein the spiral channel circumscribes an axis, and wherein the opening is configured to allow the anchor driver to advance each anchor out of the loading site distally along the axis.

15. The system according to claim 13, wherein for each anchor the anchor driver is configured to transluminally anchor the anchor to the tissue by, while the rod remains extended through the loading site, advancing the drive head and the anchor distally through the opening, out of the anchor-storage device, and transluminally to the tissue.

16. The system according to claim 15, wherein:
the system further comprises a catheter, coupled to the housing and extending distally away from the housing, and
the anchor driver is configured to transluminally anchor the anchor to the tissue by, while the rod remains extended through the loading site, advancing the drive head and the anchor distally through the opening, out of the anchor-storage device, and through the catheter to the tissue.

17. The system according to claim 16, wherein the system further comprises a catheter control handle, adapted to facilitate transluminal steering of the catheter to the tissue.

18. The system according to claim 17, wherein the catheter control handle is positioned axially between the anchor-storage device and the catheter.

19. The system according to claim 1, further comprising a series of cartridges, each cartridge of the series housing a corresponding anchor of the series, and being arranged along the spiral channel.

20. The system according to claim 19, wherein the anchor driver is configured to, for each anchor:
while the anchor is disposed in the loading site, engage the anchor using the drive head while the anchor is housed by the corresponding cartridge, and
while the drive head remains engaged with the anchor, transluminally advance the anchor to the tissue, leaving the corresponding cartridge in the anchor-storage device.
